(19) United States
(12) Patent Application Publication
(10) Pub. No.: US 2009/0068190 A1
(43) Pub. Date: Mar. 12, 2009

(54) ANTI-PROLIFERATIVE COMBINATIONS

(75) Inventor: Jonathan David Bortz, Saint Louis, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 12/199,503

(22) Filed: Aug. 27, 2008

Related U.S. Application Data
(60) Provisional application No. 60/968,695, filed on Aug. 29, 2007.

(51) Int. Cl. A61K 31/404; A61K 31/714; A61K 31/59; A61K 33/00; A61K 33/24; A61K 33/34; A61K 33/04; A61K 33/18; A61K 33/26; A61K 33/32; A61K 33/42; A61K 33/30; A61K 39/395; A61K 31/519; A61P 35/00

(52) U.S. Cl. 424/141.1; 514/415; 514/52; 514/167; 424/682; 424/655; 424/638; 424/702; 424/667; 424/646; 424/639; 424/601; 424/641; 514/249

(57) ABSTRACT

Combinations of a polyunsaturated fatty acid and diindolylmethane and pharmaceutical composition containing such combinations. Further provided are combinations including a polyunsaturated fatty acid, diindolylmethane, and folic acid. Also provided are combinations including diindolylmethane and folic acid. Also provided is a method for increasing the number of normal cells and decreasing the number of aberrant cells in a subject having the steps of administering to the subject a combination comprising a polyunsaturated fatty acid and diindolylmethane.

This application claims the benefit of U.S. provisional patent application Ser. No. 60/968,695, filed on Aug. 29, 2007, the entire disclosure of which is incorporated by reference herein.

### FIELD OF THE INVENTION

The present invention generally relates to anti-proliferative combinations. In particular, the anti-proliferative combinations are formulated into pharmaceutical compositions that may be utilized to enhance apoptotic activity in aberrant or damaged cells.

### BACKGROUND OF THE INVENTION

Several epidemiological and animal studies have identified estrogen exposure as a risk factor for cancers of the breast, endometrium, ovary, prostate, testis, and thyroid. Long-term studies of women undergoing hormone replacement therapy (HRT) have additionally found that estrogen exposure is associated with an increased risk of coronary heart disease, strokes, pulmonary embolism, altered liver function, blood clots, deep vein thrombosis, and dementia. But subsequent studies indicate that the risk factors associated with HRT depend on the particular compound and delivery method used. The adverse cardiovascular outcomes may only apply to oral dosing with progestin and equine estrogens, while other types of HRT such as topical estradiol and estriol may not produce the same risks. One overall conclusion that emerges from the series of HRT studies is that the effects of exogenous hormones clearly depend on the particular compound, and by extension, the particular metabolic pathways of these hormones.

Estrogens (estradiol, estriol, and estrone) are produced by the conversion of androgens, mediated by the enzyme aromatase, in the ovaries, and to a lesser extent in other tissues such as liver, adrenal glands, breasts, and fat. The metabolism of estrogen takes place primarily in the liver through Phase I (hydroxylation) and Phase II (methylation, glucuronidation, and sulfation) pathways, the metabolites of estrogen are ultimately excreted in the urine and feces. The intermediate metabolites of estrogen vary greatly in biological activity, and the ultimate biologic effect of estrogen depends on how it is metabolized. The major Phase I metabolic pathway for endogenous estrogens is hydrolization, which is mediated by a number of cytochrome P-450 (CYP) enzymes, present in the liver, breast, uterus, ovary and skin. Hydrolyzation takes place at three primary sites on the estrogen molecule: the 2 carbon (C-2) position yielding 2-hydroxyestrone (2-OH), the 16 carbon (C-18) position yielding 16-hydroxyestrone (16-OH), and to a much lesser extent, at the 4 carbon (C-4) position yielding 4-hydroxyestrone (4-OH). The 2-OH metabolite confers very weak estrogenic activity, while the 16-OH and 4-OH metabolites retain strong estrogenic activity and promote tissue proliferation. Numerous studies indicate that women who metabolize a larger proportion of estrogen via the C-16 and C-4 hydroxylation pathways may be at a significantly elevated risk of breast cancer, systemic lupus erythematosus, uterine cancer, and cervical dysplasia compared with women who metabolize estrogen predominantly via the C-2 pathway. The key Phase II pathway for the estrogen metabolites is glucuronidation, in which glucuronic acid is conjugated with the estrogen metabolites to facilitate its elimination from the body.

The enzymes that mediate the synthesis and metabolism of estrogen have been shown in numerous studies to be highly sensitive to hormonal status, dietary intake, and exposure to environmental toxins. The Western diet, which is high in fat, meat, and processed sugar, and low in vegetable matter, has been associated with higher risks of cancer, heart disease, diabetes, and a host of other diseases. Clearly, there are strong dietary influences on the risk of many diseases. Numerous studies have identified multiple pathways and factors involved in inducing a cell with normal physiology to undergo the metabolic shift to a highly proliferative cancer cell. A need exists for a treatment that addresses these multiple pathways and factors in a coordinated way to prevent the onset of disease, or, if cancer is induced, to selectively eliminate the highly proliferative cancer cells.

### DETAILED DESCRIPTION OF THE INVENTION

The present invention generally provides compositions formulated in a manner to enhance apoptotic activity in aberrant or damaged cells. In addition, the combinations beneficially alter the metabolism of hormones, such as androgens and estrogen. In particular, the combinations alter the metabolism of estrogen such that there is an increase in the metabolism of estrogen through the 2-hydroxy pathway, and a decrease in estrogen metabolism through the 16-hydroxy pathway and the 4-hydroxy pathway. In this manner, the compositions of the invention provide a means to treat and/or prevent indications resulting from proliferation of aberrant or damaged cells. These indications include, for example, estrogen-mediated diseases, inflammation, cardiovascular disease, cerebrovascular disease, viral infections, arthritis (e.g., osteoarthritis and rheumatoid arthritis), and several types of cancer. In various iterations of the invention, the combinations also provide a means to substantially enhance lipid peroxidation processes, augment P450 activity, and reduce the expression of FAS proteins.

### (I) Combinations

Suitable combinations generally include diindolylmethane and one or more agents selected from the group consisting of a polyunsaturated fatty acid, folate, a vitamin other than folate, a mineral, a non-steroidal anti-inflammatory agent, a steroidal anti-inflammatory agent, thiazolidinedione, an HMG-CoA reductase inhibitor, an anti-proliferative agent, an anti-immune agent, an apoptotic agent, and a hormone. Each of these ingredients is described in detail below.

### (a) Diindolylmethane

In each embodiment, the combination includes diindolylmethane (DIM). DIM is a bioactive compound with indole rings resulting from the acid condensation of indole-3-carbinol (I3C). DIM has the following chemical structure:

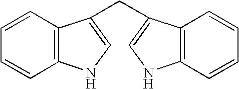

Exemplary combinations include forms of DIM that are highly bioavailable. One preferred form of DIM that is highly bioavailable is sold under the trade name Bioresponse DIM® (BioResponse, LLC, Boulder, Colo.), and is disclosed, for example, in U.S. Pat. No. 6,086,915, which is hereby incorporated by reference in its entirety.

The combinations generally include DIM in an amount from about 1 mg to about 10,000 mg. In other embodiments, the amount of DIM may be from about 1 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, about 6500 mg to about 7000 mg, about 7000 mg to about 7500 mg, about 7500 mg to about 8000 mg, about 8000 mg to about 8500 mg, about 8500 mg to about 9000 mg, about 9000 mg to about 9500 mg, about 9500 mg to about 10,000 mg, or greater than about 10,000 mg. In an alternative embodiment, the amount of DIM in the combination may range from about 25 mg to about 750 mg, from about 100 mg to about 500 mg, from about 150 mg to about 450 mg, or from about 200 mg to about 300 mg. In other embodiments, the amount of DIM in the combination is not less than about 200 mg.

### (b) Polyunsaturated Fatty Acids

The combination may, and generally does, include one or more polyunsaturated fatty acids (PUFA). Suitable PUFAs include a long chain fatty acid with at least 18 carbon atoms and at least two carbon-carbon double bonds, generally in the cis-configuration. In an exemplary embodiment, the PUFA is an omega fatty acid.

In one embodiment, the PUFA may be an omega-3 fatty acid in which the first double bond occurs in the third carbon-carbon bond from the methyl end of the carbon chain (i.e., opposite the carboxyl acid group). Suitable examples of omega-3 fatty acids include all-cis 7,10,13-hexadecatrienoic acid; all-cis-9,12,15-octadecatrienoic acid (alpha-linolenic acid, ALA); all-cis-6,9,12,15-octadecatetraenoic acid (stearidonic acid); all-cis-8,11,14,17-eicosatetraenoic acid (eicosatetraenoic acid); all-cis-5,8,11,14,17-eicosapentaenoic acid (eicosapentaenoic acid, EPA); all-cis-7,10,13,16,19-docosapentaenoic acid (clupanodonic acid, DPA); all-cis-4,7,10,13,16,19-docosahexaenoic acid (docosahexaenoic acid, DHA); all-cis-4,7,10,13,16,19-docosahexaenoic acid; and all-cis-6,9,12,15,18,21-tetracosenoic acid (nisinic acid). In an exemplary embodiment, the omega-3 fatty acid is ALA, DHA, EPA, DPA, and combinations thereof.

In an alternative embodiment, the PUFA may be an omega-6 fatty acid in which the first double bond occurs in the sixth carbon-carbon bond from the methyl end of the carbon chain. Examples of omega-6 fatty acids include all-cis-9,12-octadecadienoic acid (linoleic acid); all-cis-6,9,12-octadecatrienoic acid (gamma-linolenic acid, GLA); all-cis-11,14-eicosadienoic acid (eicosadienoic acid); all-cis-8,11,14-eicosatrienoic acid (dihomo-gamma-linolenic acid, DGLA); all-cis-5,8,11,14-eicosatetraenoic acid (arachidonic acid, AA); all-cis-13,16-docosadienoic acid (docosadienoic acid); all-cis-7,10,13,16-docosatetraenoic acid (adrenic acid); and all-cis-4,7,10,13,16-docosapentaenoic acid (docosapentaenoic acid).

In yet another alternative embodiment, the PUFA may be an omega-9 fatty acid in which the first double bond occurs in the ninth carbon-carbon bond from the methyl end of the carbon chain, or a conjugated fatty acid, in which at least one pair of double bonds are separated by only one single bond. Suitable examples of omega-9 fatty acids include cis-9-octadecenoic acid (oleic acid); cis-11-eicosenoic acid (eicosenoic acid); all-cis-5,8,11-eicosatrienoic acid (mead acid); cis-13-docosenoic acid (erucic acid), and cis-15-tetracosenoic acid (nervonic acid). Examples of conjugated fatty acids include 9Z,11E-octadeca-9,11-dienoic acid (rumenic acid); 10E,12Z-octadeca-9,11-dienoic acid; 8E,10E,12Z-octadecatrienoic acid (α-calendic acid); 8E,10E,12E-octadecatrienoic acid (β-Calendic acid); 8E,10Z,12E-octadecatrienoic acid (jacaric acid); 9E,11E,13Z-octadeca-9,11,13-trienoic acid (α-eleostearic acid); 9E,11E,13E-octadeca-9,11,13-trienoic acid (β-eleostearic acid); 9Z,11Z,13E-octadeca-9,11,13-trienoic acid (catalpic acid), and 9E,11Z,13E-octadeca-9,11,13-trienoic acid (punicic acid).

The combination may include PUFA in an amount ranging from about 1 mg to about 10,000 mg. In other embodiments, the amount of PUFA may be from about 1 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, about 6500 mg to about 7000 mg, about 7000 mg to about 7500 mg, about 7500 mg to about 8000 mg, about 8000 mg to about 8500 mg, about 8500 mg to about 9000 mg, about 9000 mg to about 9500 mg, about 9500 mg to about 10,000 mg, or greater than about 10,000 mg. In an alternative embodiment, the amount of PUFA may be from about 50 mg to about 3000 mg, from about 100 mg to about 2000 mg, from about 200 to about 1000 mg, or from about 300 mg to about 750 mg. In another alternative embodiment, the amount of PUFA is not less than about 500 mg.

### (c) Folate

Suitable folate compounds for use in the combinations include folic acid and folate (the anion form), both forms of the water-soluble Vitamin B9, as well as L-methylfolate, and methyltetrahydrofolate. An exemplary form of folate is folic acid.

The combinations may include folic acid in an amount from about 0.1 mg to about 15 mg. In other embodiments, the amount of folic acid may be about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or greater than about 15 mg. In other embodiments, the amount of folic acid may range from about 0.2 mg to about 15 mg, from about 0.4 mg to about 10 mg, from about 0.8 mg to about 5 mg, or from about 1 mg to about 2.5 mg. In other embodiments, the amount of folic acid is not less than about 1 mg.

### (d) Vitamins

The combination may include one or more vitamins in addition to folate. Suitable vitamins for use in the combinations include vitamin C, vitamin A, vitamin E, vitamin B12, vitamin K, menaquinone, menatetrenone, phylloquinone, riboflavin, niacin, vitamin D, vitamin B6, pyridoxine, thiamine, pantothenic acid, and biotin. The form of the vitamin may include salts of the vitamin, derivatives of the vitamin, compounds having the same or similar activity of a vitamin, and metabolites of a vitamin.

The combination may include one or more forms of an effective amount of any of the vitamins described herein or otherwise known in the art. An “effective amount” of a vitamin typically quantifies an amount at least about 10% of the United States Recommended Daily Allowance (“RDA”) of that particular vitamin for a subject. It is contemplated, however, that amounts of certain vitamins exceeding the RDA may be beneficial for certain subjects. For example, the amount of a given vitamin may exceed the applicable RDA by 100%, 200%, 300%, 400% or 500% or 1000% or more.

### (e) Minerals

The combination may include one or more minerals or mineral sources. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

Suitable forms of zinc, include zinc chelates (complexes of zinc and amino acids, dipeptides, or polypeptides), zinc acetate, zinc aspartate, zinc citrate, zinc glucoheptonate, zinc gluconate, zinc glycerate, zinc picolinate, zinc monomethionine and zinc sulfate.

Examples of suitable forms of copper include copper chelates, cupric oxide, copper gluconate, copper sulfate, and copper amino acid chelates.

Suitable forms of calcium include calcium alpha-ketoglutarate, calcium acetate, calcium alginate, calcium ascorbate, calcium aspartate, calcium caprylate, calcium carbonate, calcium chelates, calcium chloride, calcium citrate, calcium citrate malate, calcium formate, calcium glubionate, calcium glucoheptonate, calcium gluconate, calcium glutarate, calcium glycerophosphate, calcium lactate, calcium lysinate, calcium malate, calcium orotate, calcium oxalate, calcium oxide, calcium pantothenate, calcium phosphate, calcium pyrophosphate, calcium succinate, calcium sulfate, calcium undecylenate, coral calcium, dicalcium citrate, dicalcium malate, dihydroxycalcium malate, dicalcium phosphate, and tricalcium phosphate.

In an exemplary formulation, the combination generally will include iron. A variety of suitable forms of iron may be included. In one embodiment, the iron may be in the form of chelates, such as Ferrochel™ (Albion International, Inc., Clearfield, Utah) a commercially available bis-glycine chelate of iron, and Sumalate™ (Albion International, Inc., Clearfield, Utah) a commercially available ferrous asparto glycinate. For example, amino acid chelates are becoming well accepted as a means of increasing the metal content in biological tissues of subjects. Amino acid chelates are products resulting from the reaction of a polypeptide, dipeptide or naturally occurring alpha amino acid with a metal ion having a valence of two or more. The alpha amino acid and metal ion form a ring structure wherein the positive electrical charges of the metal ion are neutralized by the electrons of the carboxylate or free amino groups of the alpha amino acid. Although the term amino acid as used herein refers only to products obtainable through protein hydrolysis, synthetically produced amino acids are not to be excluded provided they are the same as those obtained through protein hydrolysis. Accordingly, protein hydrolysates such as polypeptides, dipeptides and naturally occurring alpha amino acids are collectively referred to as amino acids. Additional suitable amino acid chelates include for example but are not limited to ethylenediaminetetraacetic acid (EDTA), monohydroxyethylethylenediaminetriacetic acid, diethylenetriaminepentaacetic acid, monohydroxyethyldiglycine and dihydroxyethylglycine.

Other suitable forms of iron for purposes of the present invention include, for example, but are not limited to soluble iron salts, slightly soluble iron salts, insoluble iron salts, chelated iron, iron complexes, non-reactive iron such as carbonyl iron and reduced iron, and combinations thereof.

Suitable chelated iron complexes are disclosed in U.S. Pat. Nos. 4,599,152 and 4,830,716, each incorporated herein by reference.

Examples of suitable soluble iron salts include but are not limited to ferric hypophosphite, ferric albuminate, ferric chloride, ferric citrate, ferric oxide saccharate, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

Examples of suitable slightly soluble iron salts include but are not limited to ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

Suitable examples of insoluble iron salts include but are not limited to ferric sodium pyrophosphate, ferrous carbonate, ferric hydroxide, ferrous oxide, ferric oxyhydroxide, ferrous oxalate, other pharmaceutically acceptable iron salts and combinations thereof.

Examples of suitable iron complexes include but are not limited to polysaccharide-iron complex, methylidine-iron complex, ethylenediaminetetraacetic acid (EDTA)-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, N-methyl-D-glucamine dithiocarbamate (MGD)-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-ethylenediaminetetraacetic acid (EDITA) ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof.

The combination may include one or more forms of an effective amount of any of the minerals described herein or otherwise known in the art. An “effective amount” of a mineral typically quantifies an amount at least about 10% of the United States Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 100%, 200%, 300%, 400% or 500% or 1000% or more. Typically, the amount of mineral included in the combination may range from about 1 mg to about 1500 mg, about 5 mg to about 500 mg, or from about 150 mg to about 500 mg per dosage.

### (f) Non-Steroidal Anti-Inflammatory Drug

Optionally, the combination may include a non-steroidal anti-inflammatory drug (NSAID). In some embodiments, the NSAID may be a salicylate, an arylalkanoic acid, a 2-arylpropionic acid (profen), an N-arylanthranilic acid, a pyrazolidine derivative, an oxicam, a cyclooxygenase-2 (COX-2) inhibitor, and a sulphonanilide. Examples of salicylates include aspirin, amoxiprin, benorilate, choline magnesium salicylate, diflunisal, faislamine, methyl salicylate, and salicyl salicylate. Suitable examples of arylalkanoic acids include diclofenac, aceclofenac, acemetacin, bromfenac, etodolac, indometacin, nabumetone, sulindac, and tolmetin. Examples of 2-arylpropionic acids (profens) include ibuprofen, carprofen, fenbufen, fenoprofen, flurbiprofen, ketoprofen, ketorolac, loxoprofen, naproxen, tiaprofenic acid, and suprofen. Suitable examples of N-arylanthranilic acids include mefenamic acid and meclofenamic acid. Examples of pyrazolidine derivatives include phenylbutazone, azapropazone, metamizole, oxyphenbutazone, and sulfinprazone. Non-limiting examples of oxicams include piroxicam, lornoxicam, meloxicam, and tenoxicam. Suitable examples of COX-2 inhibitors include celecoxib, etoricoxib, lumiracoxib, parecoxib, rofecoxib, and valdecoxib. An example of sulphonanilide includes nimesulide.

The combinations may include NSAIDs in an amount ranging from about 1 mg to about 1500 mg. In other embodiments, the amount of NSAID in the combinations may be from about 1 mg to about 100 mg, from about 100 mg to about 200 mg, from about 200 mg to about 300 mg, from about 300 mg to about 400 mg, from about 400 mg to about 500 mg, from about 500 mg to about 600 mg, from about 600 mg to about 700 mg, from about 700 mg to about 800 mg, from about 800 mg to about 900 mg, from about 900 mg to about 1000 mg, from about 1000 mg to about 1100 mg, from about 1100 mg to about 1200 mg, from about 1200 mg to about 1300 mg, from about 1300 mg to about 1400 mg, from about 1400 mg to about 1500 mg, or greater than about 1500 mg.

Examples of dosages for exemplary NSAIDs are: from about 100 mg to about 500 mg of naproxen; from about 350 mg to about 800 mg of naproxen sodium; from about 75 mg to about 150 mg of indomethacin; from about 600 mg to about 3200 mg of ibuprofen; from about 200 mg to about 300 mg of flurbiprofen; from about 100 mg to about 500 mg of ketoprofen; from about 1200 mg to about 1800 mg of oxaprozin; from about 800 mg to about 1200 mg of etodolac; from about 10 mg to about 40 mg of ketorolac; from about 1500 mg to about 1200 mg of nabumetone; from about 100 mg to about 1000 mg of mefenamic; from about 300 mg to about 400 mg of meclofenamate sodium; from about 10 mg to about 20 mg of piroxicam to establish a plasma concentration of greater than about 5 to 6 μg/ml from about 325 mg to about 1000 mg of aspirin, and from about 325 mg to about 1000 mg of acetaminophen per dosage.

### (g) Steroids

The combination may include a steroid. Suitable non-limiting examples of steroids include dexamethasone, hydrocortisone, cortisone acetate, prednisone, prednisolone, methylprednisolone, betamethasone, triamcinolone, beclometasone, fludrocortisone acetate, deoxycorticosterone acetate, and aldosterone.

Typically, the amount of steroid in the combination may range from about 0.1 mg to about 1500 mg. In other embodiments, the amount of steroid may be from about 1 mg to about 250 mg, from about 250 mg to about 500 mg, from about 500 mg to about 750 mg, from about 750 mg to about 1000 mg, from about 1000 mg to about 1250 mg, from about 1250 mg to about 1500 mg, or greater than about 1500 mg. Examples of exemplary dosages for particular steroids are: from about 0.5 mg to about 2 mg dexamethasone; from about 20 mg to about 400 mg hydrocortisone; up to about 37.5 mg of cortisone acetate; from about 7.5 mg to about 50 mg prednisone; up to about 40 mg prednisolone; up to about 40 mg methylprednisolone; about 25 mg betamethasone; from about 0.1 mg to about 0.5 mg fludrocortisone acetate per dosage.

### (h) PPARγ Ligand

Optionally, the combination may include a PPARγ ligand. An exemplary PPARγ ligand is thiazolidinedione (TZD). Examples of TZD include, but are not limited to, exenatide, muraglitizar, and naveglitizar.

The combination may include TZD in an amount ranging from about 10 μg to about 1000 mg. In other embodiments, the amount of TZD may be from about 1 mg to about 25 mg, about 25 mg to about 50 mg, about 50 mg to about 75 mg, about 75 mg to about 100 mg, about 100 mg to about 125 mg, about 125 mg to about 150 mg, from about 150 mg to about 175 mg, from about 175 mg to about 200 mg, about 200 mg to about 225 mg, about 225 mg to about 250 mg, or greater than about 250 mg. Examples of exemplary dosages for particular TZDs are: from about 15 mg to about 45 mg of exenatide, and from about 1.5 mg to about 5 mg muraglitizar per dosage.

### (i) HMG-CoA Reductase Inhibitor

The combination may include an HMG-CoA reductase inhibitor. In some embodiments, the HMG-CoA reductase inhibitor may be naturally occurring. Non-limiting examples of naturally occurring HMG-CoA reductase inhibitors include mevastatin and compactin. Alternatively, the HMG-CoA reductase inhibitor may be synthetic. Examples of synthetic HMG-CoA reductase inhibitors include atorvastatin, fluvastatin, pitavastatin, and rosuvastatin. In an alternative embodiment, the HMG-CoA reductase inhibitor may be a synthetic derivative of a fermentation product. Examples of HMG-CoA reductase inhibitors that are synthetic derivatives of fermentation products include simvastatin, and lovastatin, pravastatin.

Generally, the combination may include an HMG-CoA reductase inhibitor in an amount ranging from about 1 mg to about 250 mg. In certain embodiments, the amount may be about 1 mg to about 25 mg, about 25 mg to about 50 mg, about 50 mg to about 75 mg, about 75 mg to about 100 mg, about 100 mg to about 125 mg, about 125 mg to about 150 mg, about 150 mg to about 175 mg, about 175 mg to about 200 mg, or greater than about 200 mg. Examples of exemplary dosages for particular HMG-CoA reductase inhibitors are: from about 10 mg to about 80 mg atorvastatin; about 1 mg to about 2 mg of pitavastatin; about 5 mg to about 40 mg of rosuvastatin; about 5 mg to about 80 mg of simvastatin; about 10 mg to about 80 mg of lovastatin; and about 10 mg to about 80 mg of pravastatin.

### (j) Anti-Proliferative Agent

The combinations may optionally include an anti-proliferative agent. In some embodiments, the anti-proliferative agent may act by a mechanism selected from the group consisting of: disruption of cellular microtubule structures, downregulation of angiogenesis, VEGF inhibition, disruption of DNA replication, alkylation, disruption of endocrine function, inhibition of tyrosine kinase, inhibition of topoisomerase, inhibition of testosterone signals, inhibition of estrogen production, inhibition of estrogen signals, and upregulation of gonadotropin-releasing hormone. Examples of agents that disrupt cellular microtubule structures include taxanes such as paclitaxel and docetaxel, and Vinca alkaloids such as vincristine, vinblastine, vinorelbine, and vindesine. Examples of agents that downregulate angiogenesis include VEGF inhibitors such as bevacizumab, and tyrosine kinase inhibitors such as imatnib, imatnib mesylate, gefitinib, and erlotinib. Examples of agents that disrupt of DNA replication include alkylation agents such as cisplatin, carboplatin, oxaliplatin, mechloethamine, cyclophosphamide, and chlorambucil; anti-metabolites such as azathioprine and mercaptopurine, Type I topoisomerase inhibitors such as irinotecan and topotecan hydrochloride, Type II topoisomerase inhibitors such as amsacrine, etoposide, etoposide phosphate, and teniposide. Examples of agents that disrupt endocrine function include. Examples of topoisomerase inhibitors include testosterone signal inhibitors such as finasteride, estrogen production inhibitors such as anastrozole, formestane, exemestane, vorozole, and letrozole, estrogen signal inhibitors such as tamoxifen, and gonadotropin-releasing hormone agonists such as goserelin.

The combination may include an anti-proliferative agent in an amount ranging from about 1 mg to about 1000 mg. In other embodiments, the amount of anti-proliferative agent may be from about 1 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1000 mg, or greater than about 1000 mg. Examples of preferred dosages for particular anti-proliferative agents are: about 250 mg paclitaxel, about 100 mg to about 150 mg docetaxel, about 10 mg vinblastine, about 100 mg vinorelbine, about 100 mg to about 400 mg bevacizumab, about 400 mg imatnib, about 50 mg to about 600 mg imatnib mesylate, about 250 mg to about 500 mg gefitnib, about 25 mg to about 150 mg erlotinib, about 50 mg cisplatin, about 100 mg to about 125 mg oxaliplatin, about 4 mg to about 8 mg mechlorethamine, about 50 mg to about 200 mg cyclophosphamide, about 4 mg to about 10 mg chlorambucil, about 50 mg to about 300 mg azathioprine, about 50 mg to about 200 mg mercaptopurine, about 100 mg to about 200 mg irinothecan, about 2 mg topotecan hydrochloride, about 75 mg to about 150 mg etoposide, about 1 mg to about 5 mg finasteride, about 25 mg to about 200 mg exemastane, about 2.5 mg to about 30 mg letrozole, and about 20 mg to about 40 mg of tamoxifen.

### (k) Anti-Immune Agent

In additional embodiments, the combination may include an anti-immune agent. In some embodiments, the anti-immune agents may be an immunosuppressant, a monoclonal antibody, and/or a cytokine. Examples of immunosuppressants include dactinomycin. Suitable examples of monoclonal antibodies include antibodies against erbB2 receptors such as trastuzumab, and antibodies against CD20 receptors such as rituximab. Examples of cytokines include interferon alpha (IFN-α), interleukin-2 (IL-2), receptor tyrosine kinase (RTK) inhibitors such as sorafenib and sunitinib, and an mTOR kinase inhibitor such as temsirolimus.

Typically, the combination may include an amount of anti-immune agent ranging from about 1 μg to about 1500 mg. In some embodiments, the amount may range from about 1 mg to about 200 mg, about 200 mg to about 400 mg, about 400 mg to about 600 mg, about 600 mg to about 800 mg, about 800 mg to about 1000 mg, about 1000 mg to about 1200 mg, about 1200 to about 1400 mg, or greater than about 1400 mg. Examples of preferred dosages for particular anti-immune agents are: about 600 μg to about 900 μg dactinomycin, about 50 mg to about 150 mg trastuzumab, about 10 mg rituximab, about 300 mg to about 600 mg cetuximab, about 200 mg to about 800 mg bevacizumab, about 50 μg interferon alpha, about 2,400,000 IU IL-2, about 12.5 mg to about 50 mg sunitinib, and about 25 mg to about 250 mg temsirolimus per dosage.

### (l) Apoptotic Agent

The combination may include an apoptotic agent. In some embodiments, the apoptotic agent may be a proteasome inhibitor, such as bortezomib. Alternatively, the apoptotic agent may be a DR4/DR5 receptor agonist. Examples of DR4/DR5 receptor agonists include recombinant human Apo2L/TRAIL.

The combination may include an apoptotic agent in an amount ranging from about 1 mg to about 50 mg. In other embodiments, the amount may range from about 1 mg to about 10 mg, about 10 mg to about 20 mg, about 20 mg to about 30 mg, about 30 mg to about 40 mg, 40 mg to about 50 mg, or greater than about 50 mg. One example of a preferred dosage for a particular anti-immune agent is about 2 mg bortezomib per dosage.

### (m) Hormones

Optionally, the combination may include a hormone. The hormone may be a predominantly female hormone or a predominantly male hormone. Suitable examples of predominantly female hormones include estrogen, conjugated estrogens, dienestrol, estrone, esterified estrogens, estradiol, estriol, estropipate, and ethinyl estradiol. Non-limiting examples of predominantly male hormones include testosterone, dehydroepiandrosterone, androstenedione, androstenediol, androsterone, and dihydrotestosterone.

The combination may include a hormone in an amount ranging from about 0.1 mg to about 1000 mg. In other embodiments, the amount may range from about 1 mg to about 10 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1000 mg, or greater than about 1000 mg. Examples of preferred dosages for particular hormones are: about 1 mg of estradiol, about 0.3 mg to about 30 mg conjugated estrogens, and about 2.5 mg to about 5 mg testosterone per dosage.

### (n) Excipients

In certain embodiments, the combinations may be formulated as pharmaceutical compositions (i.e., as detailed in section (III)) by adding one or more excipients to any of the ingredients detailed in (I)(a) to (m). A variety of commonly used excipients in pharmaceutical formulations may be selected on the basis of compatibility with the pharmaceutically active agents, and the release profile properties of the desired dosage form. For combinations comprising one or more polyunsaturated fatty acids and at least one other active ingredient, typically excipients are selected that allow large dosages of active ingredients to be contained in a single dosage formulation. Selection of certain excipients over others which may have otherwise similar chemical and physical properties may allow one or more active ingredients and at least one polyunsaturated fatty acid to be present in a single dosage form where the amount of polyunsaturated fatty acid is at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1000 mg, at least 1050 mg, at least 1100 mg, at least 1150 mg, at least 1200 mg, at least 1250 mg, at least 1300 mg, at least 1350 mg, at least 1400 mg, at least 1450 mg, or greater than about 1500 mg.

Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agent. Each excipient is described below.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In another embodiment, the excipient may be a filler. Suitable fillers include carbohydrates, inorganic compounds, and polyvinilpirrolydone. By way of non-limiting example, the filler may be calcium sulfate, both di- and tri-basic, starch, calcium carbonate, magnesium carbonate, microcrystalline cellulose, dibasic calcium phosphate, tricalcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another embodiment, the excipient may be an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

In another embodiment, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the embodiment, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present invention depending on the embodiment.

The excipient may include a taste-masking agent. Taste-masking materials include, e.g., cellulose hydroxypropyl ethers (HPC) such as Klucel®, Nisswo HPC and PrimaFlo HP22; low-substituted hydroxypropyl ethers (L-HPC), cellulose hydroxypropyl methyl ethers (HPMC) such as Seppifilm-LC, Pharmacoat.RTM., Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel.RTM., Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking materials contemplated are those described in U.S. Pat. Nos. 4,851,226, 5,075,114, and 5,876,759, each of which is hereby incorporated by reference in its entirety.

In various embodiments, the excipient may include a pH modifier. In certain embodiments, the pH modifier may include sodium carbonate or sodium bicarbonate. In other embodiments, an antioxidant such as BHT or BHA is utilized.

The weight fraction of the excipient or combination of excipients in the pharmaceutical composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

### (II) Exemplary Formulations

Any of the ingredients detailed in I(a) to (m) may be combined together to form a suitable combination of the invention. By way of non-limiting example, useful combinations include the following: (1) at least one polyunsaturated fatty acid and diindolylemethane; (2) at least one polyunsaturated fatty acid, diindolylmethane, and a vitamin; (3) at least one polyunsaturated fatty acid, diindolylmethane, and a mineral; (4) at least one polyunsaturated fatty acid, diindolylmethane, and a NSAID; (5) at least one polyunsaturated fatty acid, diindolylmethane, and a steroid; (6) at least one polyunsaturated fatty acid, diindolylmethane, and TZD; (7) at least one polyunsaturated fatty acid, diindolylmethane, and an HMG-CoA reductase inhibitor; (8) at least one polyunsaturated fatty acid, diindolylmethane, and an anti-proliferative agent; (9) at least one polyunsaturated fatty acid, diindolylmethane, and an anti-immune agent; (10) at least one polyunsaturated fatty acid, diindolylmethane, and an apoptotic agent; (11) at least one polyunsaturated fatty acid, diindolylmethane, and a hormone; (12) at least one polyunsaturated fatty acid, diindolylmethane, and folic acid; (13) at least one polyunsaturated fatty acid, diindolylmethane, folic acid, and a vitamin; (14) at least one polyunsaturated fatty acid, diindolylmethane, folic acid, and a mineral; (15) at least one polyunsaturated fatty acid, diindolylmethane, folic acid, and a NSAID; (16) at least one polyunsaturated fatty acid, diindolylmethane, folic acid, and a steroid; (17) at least one polyunsaturated fatty acid, diindolylmethane, folic acid, and TZD; (18) at least one polyunsaturated fatty acid, diindolylmethane, folic acid, and an HMG-CoA reductase inhibitor; (19) at least one polyunsaturated fatty acid, diindolylmethane, folic acid, and an anti-proliferative agent; (20) at least one polyunsaturated fatty acid, diindolylmethane, folic acid, and an anti-immune agent; (21) at least one polyunsaturated fatty acid, diindolylmethane, folic acid, and an apoptotic agent; (22) at least one polyunsaturated fatty acid, diindolylmethane, folic acid, and a hormone; (23) diindolylmethane and TZD; (24) diindolylmethane and folate; (25) diindolylmethane, TZD, and a vitamin; (26) diindolylmethane, TZD, and a mineral; (27) diindolylmethane, TZD, and a NSAID; (28) diindolylmethane, TZD, and a steroid; (29) diindolylmethane, TZD, and an HMG-CoA reductase inhibitor; (30) diindolylmethane, TZD, and an anti-proliferative agent; (31) diindolylmethane, TZD, and an anti-immune agent; (32) diindolylmethane, TZD, and an apoptotic agent; (33) diindolylmethane, TZD, and a hormone; (34) diindolylmethane and HMG-CoA reductase inhibitor; (35) diindolylmethane, HMG-CoA reductase inhibitor, and a vitamin; (36) diindolylmethane, HMG-CoA reductase inhibitor, and a mineral; (37) diindolylmethane, HMG-CoA reductase inhibitor, and a NSAID; (38) diindolylmethane, HMG-CoA reductase inhibitor, and a steroid; (39) diindolylmethane, HMG-CoA reductase inhibitor, and an anti-proliferative agent; (40) diindolylmethane, HMG-CoA reductase inhibitor, and an anti-immune agent; (41) diindolylmethane, HMG-CoA reductase inhibitor, and an apoptotic agent; and (42) diindolylmethane, HMG-CoA reductase inhibitor, and a hormone.

One exemplary combination comprises at least one polyunsaturated fatty acid and diindolylmethane. Non-limiting examples of this combination are delineated in Table A.

TABLE A

| First Agent | Second Agent |
|---|---|
| alpha-linolenic acid | diindolylmethane |
| docosahexaenoic acid | diindolylmethane |
| gamma-linolenic acid | diindolylmethane |
| eicosapentaenoic acid | diindolylmethane |
| alpha-linolenic acid + docosahexaenoic acid | diindolylmethane |
| alpha-linolenic acid + gamma-linolenic acid | diindolylmethane |
| alpha-linolenic acid + eicosapentaenoic acid | diindolylmethane |
| docosahexaenoic acid + gamma-linolenic acid | diindolylmethane |
| docosahexaenoic acid + eicosapentaenoic acid | diindolylmethane |
| gamma-linolenic acid + eicosapentaenoic acid | diindolylmethane |
| alpha-linolenic acid + docosahexaenoic acid + | diindolylmethane |
| gamma-linolenic acid | |
| alpha-linolenic acid + docosahexaenoic acid + | diindolylmethane |
| eicosapentaenoic acid | |
| docosahexaenoic acid + gamma-linolenic acid + | diindolylmethane |
| eicosapentaenoic acid | |
| alpha-linolenic acid + gamma-linolenic acid + | diindolylmethane |
| eicosapentaenoic acid | |
| alpha-linolenic acid + docosahexaenoic acid + | diindolylmethane |
| gamma-linolenic acid + eicosapentaenoic acid | |

In one alternative exemplary embodiment, folic acid may be combined with any of the combinations delineated in Table A.

Yet another exemplary combination comprises at least one polyunsaturated fatty acid, diindolylmethane, and a NSAID. Non-limiting examples of this combination are delineated in Table B.

TABLE B

| First Agent | Second Agent | Third Agent |
|---|---|---|
| alpha-linolenic acid | diindolylmethane | aspirin |
| alpha-linolenic acid | diindolylmethane | amoxiprin |
| alpha-linolenic acid | diindolylmethane | benorilate |
| alpha-linolenic acid | diindolylmethane | choline magnesium |
| | | salicylate |
| alpha-linolenic acid | diindolylmethane | diflunisal |
| alpha-linolenic acid | diindolylmethane | faislamine |
| alpha-linolenic acid | diindolylmethane | methylsalicylate |
| alpha-linolenic acid | diindolylmethane | salicylsalicylate |
| alpha-linolenic acid | diindolylmethane | diclofenac |
| alpha-linolenic acid | diindolylmethane | aceclofenac |
| alpha-linolenic acid | diindolylmethane | acemetacin |
| alpha-linolenic acid | diindolylmethane | bromfenac |
| alpha-linolenic acid | diindolylmethane | etodolac |
| alpha-linolenic acid | diindolylmethane | indometacin |
| alpha-linolenic acid | diindolylmethane | nabumetone |
| alpha-linolenic acid | diindolylmethane | sulindac |
| alpha-linolenic acid | diindolylmethane | tolmetin |
| alpha-linolenic acid | diindolylmethane | ibuprofen |
| alpha-linolenic acid | diindolylmethane | carprofen |
| alpha-linolenic acid | diindolylmethane | fenbufen |
| alpha-linolenic acid | diindolylmethane | fenoprofen |
| alpha-linolenic acid | diindolylmethane | flurbiprofen |
| alpha-linolenic acid | diindolylmethane | ketoprofen |
| alpha-linolenic acid | diindolylmethane | ketorolac |
| alpha-linolenic acid | diindolylmethane | loxoprofen |
| alpha-linolenic acid | diindolylmethane | naproxen |
| alpha-linolenic acid | diindolylmethane | tiaprofenicacid |
| alpha-linolenic acid | diindolylmethane | suprofen |
| alpha-linolenic acid | diindolylmethane | mefenamicacid |
| alpha-linolenic acid | diindolylmethane | meclofenamicacid |
| alpha-linolenic acid | diindolylmethane | phenylbutazone |
| alpha-linolenic acid | diindolylmethane | azapropazone |
| alpha-linolenic acid | diindolylmethane | metamizole |
| alpha-linolenic acid | diindolylmethane | oxyphenbutazone |
| alpha-linolenic acid | diindolylmethane | sulfinprazone |
| alpha-linolenic acid | diindolylmethane | piroxicam |
| alpha-linolenic acid | diindolylmethane | lornoxicam |
| alpha-linolenic acid | diindolylmethane | meloxicam |
| alpha-linolenic acid | diindolylmethane | tenoxicam |
| alpha-linolenic acid | diindolylmethane | celecoxib |
| alpha-linolenic acid | diindolylmethane | etoricoxib |
| alpha-linolenic acid | diindolylmethane | lumiracoxib |
| alpha-linolenic acid | diindolylmethane | parecoxib |
| alpha-linolenic acid | diindolylmethane | rofecoxib |
| alpha-linolenic acid | diindolylmethane | valdecoxib |
| alpha-linolenic acid | diindolylmethane | nimesulide |
| docosahexaenoic acid | diindolylmethane | aspirin |
| docosahexaenoic acid | diindolylmethane | amoxiprin |
| docosahexaenoic acid | diindolylmethane | benorilate |
| docosahexaenoic acid | diindolylmethane | choline magnesium |
| | | salicylate |
| docosahexaenoic acid | diindolylmethane | diflunisal |
| docosahexaenoic acid | diindolylmethane | faislamine |
| docosahexaenoic acid | diindolylmethane | methylsalicylate |
| docosahexaenoic acid | diindolylmethane | salicylsalicylate |
| docosahexaenoic acid | diindolylmethane | diclofenac |
| docosahexaenoic acid | diindolylmethane | aceclofenac |
| docosahexaenoic acid | diindolylmethane | acemetacin |
| docosahexaenoic acid | diindolylmethane | bromfenac |
| docosahexaenoic acid | diindolylmethane | etodolac |
| docosahexaenoic acid | diindolylmethane | indometacin |
| docosahexaenoic acid | diindolylmethane | nabumetone |
| docosahexaenoic acid | diindolylmethane | sulindac |
| docosahexaenoic acid | diindolylmethane | tolmetin |
| docosahexaenoic acid | diindolylmethane | ibuprofen |
| docosahexaenoic acid | diindolylmethane | carprofen |
| docosahexaenoic acid | diindolylmethane | fenbufen |
| docosahexaenoic acid | diindolylmethane | fenoprofen |
| docosahexaenoic acid | diindolylmethane | flurbiprofen |
| docosahexaenoic acid | diindolylmethane | ketoprofen |
| docosahexaenoic acid | diindolylmethane | ketorolac |
| docosahexaenoic acid | diindolylmethane | loxoprofen |
| docosahexaenoic acid | diindolylmethane | naproxen |
| docosahexaenoic acid | diindolylmethane | tiaprofenicacid |
| docosahexaenoic acid | diindolylmethane | suprofen |
| docosahexaenoic acid | diindolylmethane | mefenamicacid |
| docosahexaenoic acid | diindolylmethane | meclofenamicacid |
| docosahexaenoic acid | diindolylmethane | phenylbutazone |
| docosahexaenoic acid | diindolylmethane | azapropazone |
| docosahexaenoic acid | diindolylmethane | metamizole |
| docosahexaenoic acid | diindolylmethane | oxyphenbutazone |
| docosahexaenoic acid | diindolylmethane | sulfinprazone |
| docosahexaenoic acid | diindolylmethane | piroxicam |
| docosahexaenoic acid | diindolylmethane | lornoxicam |
| docosahexaenoic acid | diindolylmethane | meloxicam |
| docosahexaenoic acid | diindolylmethane | tenoxicam |
| docosahexaenoic acid | diindolylmethane | celecoxib |
| docosahexaenoic acid | diindolylmethane | etoricoxib |
| docosahexaenoic acid | diindolylmethane | lumiracoxib |
| docosahexaenoic acid | diindolylmethane | parecoxib |
| docosahexaenoic acid | diindolylmethane | rofecoxib |
| docosahexaenoic acid | diindolylmethane | valdecoxib |
| docosahexaenoic acid | diindolylmethane | nimesulide |
| gamma-linolenic acid | diindolylmethane | aspirin |
| gamma-linolenic acid | diindolylmethane | amoxiprin |
| gamma-linolenic acid | diindolylmethane | benorilate |
| gamma-linolenic acid | diindolylmethane | choline magnesium |
| | | salicylate |
| gamma-linolenic acid | diindolylmethane | diflunisal |
| gamma-linolenic acid | diindolylmethane | faislamine |
| gamma-linolenic acid | diindolylmethane | methylsalicylate |
| gamma-linolenic acid | diindolylmethane | salicylsalicylate |
| gamma-linolenic acid | diindolylmethane | diclofenac |
| gamma-linolenic acid | diindolylmethane | aceclofenac |
| gamma-linolenic acid | diindolylmethane | acemetacin |
| gamma-linolenic acid | diindolylmethane | bromfenac |
| gamma-linolenic acid | diindolylmethane | etodolac |
| gamma-linolenic acid | diindolylmethane | indometacin |
| gamma-linolenic acid | diindolylmethane | nabumetone |
| gamma-linolenic acid | diindolylmethane | sulindac |
| gamma-linolenic acid | diindolylmethane | tolmetin |
| gamma-linolenic acid | diindolylmethane | ibuprofen |
| gamma-linolenic acid | diindolylmethane | carprofen |
| gamma-linolenic acid | diindolylmethane | fenbufen |
| gamma-linolenic acid | diindolylmethane | fenoprofen |
| gamma-linolenic acid | diindolylmethane | flurbiprofen |
| gamma-linolenic acid | diindolylmethane | ketoprofen |
| gamma-linolenic acid | diindolylmethane | ketorolac |
| gamma-linolenic acid | diindolylmethane | loxoprofen |
| gamma-linolenic acid | diindolylmethane | naproxen |
| gamma-linolenic acid | diindolylmethane | tiaprofenicacid |
| gamma-linolenic acid | diindolylmethane | suprofen |
| gamma-linolenic acid | diindolylmethane | mefenamicacid |
| gamma-linolenic acid | diindolylmethane | meclofenamicacid |
| gamma-linolenic acid | diindolylmethane | phenylbutazone |
| gamma-linolenic acid | diindolylmethane | azapropazone |
| gamma-linolenic acid | diindolylmethane | metamizole |
| gamma-linolenic acid | diindolylmethane | oxyphenbutazone |
| gamma-linolenic acid | diindolylmethane | sulfinprazone |
| gamma-linolenic acid | diindolylmethane | piroxicam |
| gamma-linolenic acid | diindolylmethane | lornoxicam |
| gamma-linolenic acid | diindolylmethane | meloxicam |
| gamma-linolenic acid | diindolylmethane | tenoxicam |
| gamma-linolenic acid | diindolylmethane | celecoxib |
| gamma-linolenic acid | diindolylmethane | etoricoxib |
| gamma-linolenic acid | diindolylmethane | lumiracoxib |
| gamma-linolenic acid | diindolylmethane | parecoxib |
| gamma-linolenic acid | diindolylmethane | rofecoxib |
| gamma-linolenic acid | diindolylmethane | valdecoxib |
| gamma-linolenic acid | diindolylmethane | nimesulide |
| eicosapentaenoic acid | diindolylmethane | aspirin |
| eicosapentaenoic acid | diindolylmethane | amoxiprin |
| eicosapentaenoic acid | diindolylmethane | benorilate |
| eicosapentaenoic acid | diindolylmethane | choline magnesium |
| | | salicylate |
| eicosapentaenoic acid | diindolylmethane | diflunisal |
| eicosapentaenoic acid | diindolylmethane | faislamine |
| eicosapentaenoic acid | diindolylmethane | methylsalicylate |
| eicosapentaenoic acid | diindolylmethane | salicylsalicylate |
| eicosapentaenoic acid | diindolylmethane | diclofenac |
| eicosapentaenoic acid | diindolylmethane | aceclofenac |
| eicosapentaenoic acid | diindolylmethane | acemetacin |
| eicosapentaenoic acid | diindolylmethane | bromfenac |
| eicosapentaenoic acid | diindolylmethane | etodolac |
| eicosapentaenoic acid | diindolylmethane | indometacin |
| eicosapentaenoic acid | diindolylmethane | nabumetone |
| eicosapentaenoic acid | diindolylmethane | sulindac |
| eicosapentaenoic acid | diindolylmethane | tolmetin |
| eicosapentaenoic acid | diindolylmethane | ibuprofen |
| eicosapentaenoic acid | diindolylmethane | carprofen |
| eicosapentaenoic acid | diindolylmethane | fenbufen |
| eicosapentaenoic acid | diindolylmethane | fenoprofen |
| eicosapentaenoic acid | diindolylmethane | flurbiprofen |
| eicosapentaenoic acid | diindolylmethane | ketoprofen |
| eicosapentaenoic acid | diindolylmethane | ketorolac |
| eicosapentaenoic acid | diindolylmethane | loxoprofen |
| eicosapentaenoic acid | diindolylmethane | naproxen |
| eicosapentaenoic acid | diindolylmethane | tiaprofenicacid |
| eicosapentaenoic acid | diindolylmethane | suprofen |
| eicosapentaenoic acid | diindolylmethane | mefenamicacid |
| eicosapentaenoic acid | diindolylmethane | meclofenamicacid |
| eicosapentaenoic acid | diindolylmethane | phenylbutazone |
| eicosapentaenoic acid | diindolylmethane | azapropazone |
| eicosapentaenoic acid | diindolylmethane | metamizole |
| eicosapentaenoic acid | diindolylmethane | oxyphenbutazone |
| eicosapentaenoic acid | diindolylmethane | sulfinprazone |
| eicosapentaenoic acid | diindolylmethane | piroxicam |
| eicosapentaenoic acid | diindolylmethane | lornoxicam |
| eicosapentaenoic acid | diindolylmethane | meloxicam |
| eicosapentaenoic acid | diindolylmethane | tenoxicam |
| eicosapentaenoic acid | diindolylmethane | celecoxib |
| eicosapentaenoic acid | diindolylmethane | etoricoxib |
| eicosapentaenoic acid | diindolylmethane | lumiracoxib |
| eicosapentaenoic acid | diindolylmethane | parecoxib |
| eicosapentaenoic acid | diindolylmethane | rofecoxib |
| eicosapentaenoic acid | diindolylmethane | valdecoxib |
| eicosapentaenoic acid | diindolylmethane | nimesulide |
| alpha-linolenic acid + docosahexaenoic acid | diindolylmethane | aspirin |
| alpha-linolenic acid + docosahexaenoic acid | diindolylmethane | amoxiprin |
| alpha-linolenic acid + docosahexaenoic acid | diindolylmethane | benorilate |
| alpha-linolenic acid + docosahexaenoic acid | diindolylmethane | choline magnesium |
| | | salicylate |
| alpha-linolenic acid + docosahexaenoic acid | diindolylmethane | diflunisal |
| alpha-linolenic acid + docosahexaenoic acid | diindolylmethane | faislamine |
| alpha-linolenic acid + docosahexaenoic acid | diindolylmethane | methylsalicylate |
| alpha-linolenic acid + docosahexaenoic acid | diindolylmethane | salicylsalicylate |
| alpha-linolenic acid + docosahexaenoic acid | diindolylmethane | diclofenac |
| alpha-linolenic acid + docosahexaenoic acid | diindolylmethane | aceclofenac |
| alpha-linolenic acid + docosahexaenoic acid | diindolylmethane | acemetacin |
| alpha-linolenic acid + docosahexaenoic acid | diindolylmethane | bromfenac |
| alpha-linolenic acid + docosahexaenoic acid | diindolylmethane | etodolac |
| alpha-linolenic acid + docosahexaenoic acid | diindolylmethane | indometacin |
| alpha-linolenic acid + docosahexaenoic acid | diindolylmethane | nabumetone |
| alpha-linolenic acid + docosahexaenoic acid | diindolylmethane | sulindac |
| alpha-linolenic acid + docosahexaenoic acid | diindolylmethane | tolmetin |
| alpha-linolenic acid + docosahexaenoic acid | diindolylmethane | ibuprofen |
| alpha-linolenic acid + docosahexaenoic acid | diindolylmethane | carprofen |
| alpha-linolenic acid + docosahexaenoic acid | diindolylmethane | fenbufen |
| alpha-linolenic acid + docosahexaenoic acid | diindolylmethane | fenoprofen |
| alpha-linolenic acid + docosahexaenoic acid | diindolylmethane | flurbiprofen |
| alpha-linolenic acid + docosahexaenoic acid | diindolylmethane | ketoprofen |
| alpha-linolenic acid + docosahexaenoic acid | diindolylmethane | ketorolac |
| alpha-linolenic acid + docosahexaenoic acid | diindolylmethane | loxoprofen |
| alpha-linolenic acid + docosahexaenoic acid | diindolylmethane | naproxen |
| alpha-linolenic acid + docosahexaenoic acid | diindolylmethane | tiaprofenicacid |
| alpha-linolenic acid + docosahexaenoic acid | diindolylmethane | suprofen |
| alpha-linolenic acid + docosahexaenoic acid | diindolylmethane | mefenamicacid |
| alpha-linolenic acid + docosahexaenoic acid | diindolylmethane | meclofenamicacid |
| alpha-linolenic acid + docosahexaenoic acid | diindolylmethane | phenylbutazone |
| alpha-linolenic acid + docosahexaenoic acid | diindolylmethane | azapropazone |
| alpha-linolenic acid + docosahexaenoic acid | diindolylmethane | metamizole |
| alpha-linolenic acid + docosahexaenoic acid | diindolylmethane | oxyphenbutazone |
| alpha-linolenic acid + docosahexaenoic acid | diindolylmethane | sulfinprazone |
| alpha-linolenic acid + docosahexaenoic acid | diindolylmethane | piroxicam |
| alpha-linolenic acid + docosahexaenoic acid | diindolylmethane | lornoxicam |
| alpha-linolenic acid + docosahexaenoic acid | diindolylmethane | meloxicam |
| alpha-linolenic acid + docosahexaenoic acid | diindolylmethane | tenoxicam |
| alpha-linolenic acid + docosahexaenoic acid | diindolylmethane | celecoxib |
| alpha-linolenic acid + docosahexaenoic acid | diindolylmethane | etoricoxib |
| alpha-linolenic acid + docosahexaenoic acid | diindolylmethane | lumiracoxib |
| alpha-linolenic acid + docosahexaenoic acid | diindolylmethane | parecoxib |
| alpha-linolenic acid + docosahexaenoic acid | diindolylmethane | rofecoxib |
| alpha-linolenic acid + docosahexaenoic acid | diindolylmethane | valdecoxib |
| alpha-linolenic acid + docosahexaenoic acid | diindolylmethane | nimesulide |
| alpha-linolenic acid + gamma-linolenic acid | diindolylmethane | aspirin |
| alpha-linolenic acid + gamma-linolenic acid | diindolylmethane | amoxiprin |
| alpha-linolenic acid + gamma-linolenic acid | diindolylmethane | benorilate |
| alpha-linolenic acid + gamma-linolenic acid | diindolylmethane | choline magnesium |
| | | salicylate |
| alpha-linolenic acid + gamma-linolenic acid | diindolylmethane | diflunisal |
| alpha-linolenic acid + gamma-linolenic acid | diindolylmethane | faislamine |
| alpha-linolenic acid + gamma-linolenic acid | diindolylmethane | methylsalicylate |
| alpha-linolenic acid + gamma-linolenic acid | diindolylmethane | salicylsalicylate |
| alpha-linolenic acid + gamma-linolenic acid | diindolylmethane | diclofenac |
| alpha-linolenic acid + gamma-linolenic acid | diindolylmethane | aceclofenac |
| alpha-linolenic acid + gamma-linolenic acid | diindolylmethane | acemetacin |
| alpha-linolenic acid + gamma-linolenic acid | diindolylmethane | bromfenac |
| alpha-linolenic acid + gamma-linolenic acid | diindolylmethane | etodolac |
| alpha-linolenic acid + gamma-linolenic acid | diindolylmethane | indometacin |
| alpha-linolenic acid + gamma-linolenic acid | diindolylmethane | nabumetone |
| alpha-linolenic acid + gamma-linolenic acid | diindolylmethane | sulindac |
| alpha-linolenic acid + gamma-linolenic acid | diindolylmethane | tolmetin |
| alpha-linolenic acid + gamma-linolenic acid | diindolylmethane | ibuprofen |
| alpha-linolenic acid + gamma-linolenic acid | diindolylmethane | carprofen |
| alpha-linolenic acid + gamma-linolenic acid | diindolylmethane | fenbufen |
| alpha-linolenic acid + gamma-linolenic acid | diindolylmethane | fenoprofen |
| alpha-linolenic acid + gamma-linolenic acid | diindolylmethane | flurbiprofen |
| alpha-linolenic acid + gamma-linolenic acid | diindolylmethane | ketoprofen |
| alpha-linolenic acid + gamma-linolenic acid | diindolylmethane | ketorolac |
| alpha-linolenic acid + gamma-linolenic acid | diindolylmethane | loxoprofen |
| alpha-linolenic acid + gamma-linolenic acid | diindolylmethane | naproxen |
| alpha-linolenic acid + gamma-linolenic acid | diindolylmethane | tiaprofenicacid |
| alpha-linolenic acid + gamma-linolenic acid | diindolylmethane | suprofen |
| alpha-linolenic acid + gamma-linolenic acid | diindolylmethane | mefenamicacid |
| alpha-linolenic acid + gamma-linolenic acid | diindolylmethane | meclofenamicacid |
| alpha-linolenic acid + gamma-linolenic acid | diindolylmethane | phenylbutazone |
| alpha-linolenic acid + gamma-linolenic acid | diindolylmethane | azapropazone |
| alpha-linolenic acid + gamma-linolenic acid | diindolylmethane | metamizole |
| alpha-linolenic acid + gamma-linolenic acid | diindolylmethane | oxyphenbutazone |
| alpha-linolenic acid + gamma-linolenic acid | diindolylmethane | sulfinprazone |
| alpha-linolenic acid + gamma-linolenic acid | diindolylmethane | piroxicam |
| alpha-linolenic acid + gamma-linolenic acid | diindolylmethane | lornoxicam |
| alpha-linolenic acid + gamma-linolenic acid | diindolylmethane | meloxicam |
| alpha-linolenic acid + gamma-linolenic acid | diindolylmethane | tenoxicam |
| alpha-linolenic acid + gamma-linolenic acid | diindolylmethane | celecoxib |
| alpha-linolenic acid + gamma-linolenic acid | diindolylmethane | etoricoxib |
| alpha-linolenic acid + gamma-linolenic acid | diindolylmethane | lumiracoxib |
| alpha-linolenic acid + gamma-linolenic acid | diindolylmethane | parecoxib |
| alpha-linolenic acid + gamma-linolenic acid | diindolylmethane | rofecoxib |
| alpha-linolenic acid + gamma-linolenic acid | diindolylmethane | valdecoxib |
| alpha-linolenic acid + gamma-linolenic acid | diindolylmethane | nimesulide |
| alpha-linolenic acid + eicosapentaenoic acid | diindolylmethane | aspirin |
| alpha-linolenic acid + eicosapentaenoic acid | diindolylmethane | amoxiprin |
| alpha-linolenic acid + eicosapentaenoic acid | diindolylmethane | benorilate |
| alpha-linolenic acid + eicosapentaenoic acid | diindolylmethane | choline magnesium |
| | | salicylate |
| alpha-linolenic acid + eicosapentaenoic acid | diindolylmethane | diflunisal |
| alpha-linolenic acid + eicosapentaenoic acid | diindolylmethane | faislamine |
| alpha-linolenic acid + eicosapentaenoic acid | diindolylmethane | methylsalicylate |
| alpha-linolenic acid + eicosapentaenoic acid | diindolylmethane | salicylsalicylate |
| alpha-linolenic acid + eicosapentaenoic acid | diindolylmethane | diclofenac |
| alpha-linolenic acid + eicosapentaenoic acid | diindolylmethane | aceclofenac |
| alpha-linolenic acid + eicosapentaenoic acid | diindolylmethane | acemetacin |
| alpha-linolenic acid + eicosapentaenoic acid | diindolylmethane | bromfenac |
| alpha-linolenic acid + eicosapentaenoic acid | diindolylmethane | etodolac |
| alpha-linolenic acid + eicosapentaenoic acid | diindolylmethane | indometacin |
| alpha-linolenic acid + eicosapentaenoic acid | diindolylmethane | nabumetone |
| alpha-linolenic acid + eicosapentaenoic acid | diindolylmethane | sulindac |
| alpha-linolenic acid + eicosapentaenoic acid | diindolylmethane | tolmetin |
| alpha-linolenic acid + eicosapentaenoic acid | diindolylmethane | ibuprofen |
| alpha-linolenic acid + eicosapentaenoic acid | diindolylmethane | carprofen |
| alpha-linolenic acid + eicosapentaenoic acid | diindolylmethane | fenbufen |
| alpha-linolenic acid + eicosapentaenoic acid | diindolylmethane | fenoprofen |
| alpha-linolenic acid + eicosapentaenoic acid | diindolylmethane | flurbiprofen |
| alpha-linolenic acid + eicosapentaenoic acid | diindolylmethane | ketoprofen |
| alpha-linolenic acid + eicosapentaenoic acid | diindolylmethane | ketorolac |
| alpha-linolenic acid + eicosapentaenoic acid | diindolylmethane | loxoprofen |
| alpha-linolenic acid + eicosapentaenoic acid | diindolylmethane | naproxen |
| alpha-linolenic acid + eicosapentaenoic acid | diindolylmethane | tiaprofenicacid |
| alpha-linolenic acid + eicosapentaenoic acid | diindolylmethane | suprofen |
| alpha-linolenic acid + eicosapentaenoic acid | diindolylmethane | mefenamicacid |
| alpha-linolenic acid + eicosapentaenoic acid | diindolylmethane | meclofenamicacid |
| alpha-linolenic acid + eicosapentaenoic acid | diindolylmethane | phenylbutazone |
| alpha-linolenic acid + eicosapentaenoic acid | diindolylmethane | azapropazone |
| alpha-linolenic acid + eicosapentaenoic acid | diindolylmethane | metamizole |
| alpha-linolenic acid + eicosapentaenoic acid | diindolylmethane | oxyphenbutazone |
| alpha-linolenic acid + eicosapentaenoic acid | diindolylmethane | sulfinprazone |
| alpha-linolenic acid + eicosapentaenoic acid | diindolylmethane | piroxicam |
| alpha-linolenic acid + eicosapentaenoic acid | diindolylmethane | lornoxicam |
| alpha-linolenic acid + eicosapentaenoic acid | diindolylmethane | meloxicam |
| alpha-linolenic acid + eicosapentaenoic acid | diindolylmethane | tenoxicam |
| alpha-linolenic acid + eicosapentaenoic acid | diindolylmethane | celecoxib |
| alpha-linolenic acid + eicosapentaenoic acid | diindolylmethane | etoricoxib |
| alpha-linolenic acid + eicosapentaenoic acid | diindolylmethane | lumiracoxib |
| alpha-linolenic acid + eicosapentaenoic acid | diindolylmethane | parecoxib |
| alpha-linolenic acid + eicosapentaenoic acid | diindolylmethane | rofecoxib |
| alpha-linolenic acid + eicosapentaenoic acid | diindolylmethane | valdecoxib |
| alpha-linolenic acid + eicosapentaenoic acid | diindolylmethane | nimesulide |
| docosahexaenoic acid + gamma-linolenic acid | diindolylmethane | aspirin |
| docosahexaenoic acid + gamma-linolenic acid | diindolylmethane | amoxiprin |
| docosahexaenoic acid + gamma-linolenic acid | diindolylmethane | benorilate |
| docosahexaenoic acid + gamma-linolenic acid | diindolylmethane | choline magnesium |
| | | salicylate |
| docosahexaenoic acid + gamma-linolenic acid | diindolylmethane | diflunisal |
| docosahexaenoic acid + gamma-linolenic acid | diindolylmethane | faislamine |
| docosahexaenoic acid + gamma-linolenic acid | diindolylmethane | methylsalicylate |
| docosahexaenoic acid + gamma-linolenic acid | diindolylmethane | salicylsalicylate |
| docosahexaenoic acid + gamma-linolenic acid | diindolylmethane | diclofenac |
| docosahexaenoic acid + gamma-linolenic acid | diindolylmethane | aceclofenac |
| docosahexaenoic acid + gamma-linolenic acid | diindolylmethane | acemetacin |
| docosahexaenoic acid + gamma-linolenic acid | diindolylmethane | bromfenac |
| docosahexaenoic acid + gamma-linolenic acid | diindolylmethane | etodolac |
| docosahexaenoic acid + gamma-linolenic acid | diindolylmethane | indometacin |
| docosahexaenoic acid + gamma-linolenic acid | diindolylmethane | nabumetone |
| docosahexaenoic acid + gamma-linolenic acid | diindolylmethane | sulindac |
| docosahexaenoic acid + gamma-linolenic acid | diindolylmethane | tolmetin |
| docosahexaenoic acid + gamma-linolenic acid | diindolylmethane | ibuprofen |
| docosahexaenoic acid + gamma-linolenic acid | diindolylmethane | carprofen |
| docosahexaenoic acid + gamma-linolenic acid | diindolylmethane | fenbufen |
| docosahexaenoic acid + gamma-linolenic acid | diindolylmethane | fenoprofen |
| docosahexaenoic acid + gamma-linolenic acid | diindolylmethane | flurbiprofen |
| docosahexaenoic acid + gamma-linolenic acid | diindolylmethane | ketoprofen |
| docosahexaenoic acid + gamma-linolenic acid | diindolylmethane | ketorolac |
| docosahexaenoic acid + gamma-linolenic acid | diindolylmethane | loxoprofen |
| docosahexaenoic acid + gamma-linolenic acid | diindolylmethane | naproxen |
| docosahexaenoic acid + gamma-linolenic acid | diindolylmethane | tiaprofenicacid |
| docosahexaenoic acid + gamma-linolenic acid | diindolylmethane | suprofen |
| docosahexaenoic acid + gamma-linolenic acid | diindolylmethane | mefenamicacid |
| docosahexaenoic acid + gamma-linolenic acid | diindolylmethane | meclofenamicacid |
| docosahexaenoic acid + gamma-linolenic acid | diindolylmethane | phenylbutazone |
| docosahexaenoic acid + gamma-linolenic acid | diindolylmethane | azapropazone |
| docosahexaenoic acid + gamma-linolenic acid | diindolylmethane | metamizole |
| docosahexaenoic acid + gamma-linolenic acid | diindolylmethane | oxyphenbutazone |
| docosahexaenoic acid + gamma-linolenic acid | diindolylmethane | sulfinprazone |
| docosahexaenoic acid + gamma-linolenic acid | diindolylmethane | piroxicam |
| docosahexaenoic acid + gamma-linolenic acid | diindolylmethane | lornoxicam |
| docosahexaenoic acid + gamma-linolenic acid | diindolylmethane | meloxicam |
| docosahexaenoic acid + gamma-linolenic acid | diindolylmethane | tenoxicam |
| docosahexaenoic acid + gamma-linolenic acid | diindolylmethane | celecoxib |
| docosahexaenoic acid + gamma-linolenic acid | diindolylmethane | etoricoxib |
| docosahexaenoic acid + gamma-linolenic acid | diindolylmethane | lumiracoxib |
| docosahexaenoic acid + gamma-linolenic acid | diindolylmethane | parecoxib |
| docosahexaenoic acid + gamma-linolenic acid | diindolylmethane | rofecoxib |
| docosahexaenoic acid + gamma-linolenic acid | diindolylmethane | valdecoxib |
| docosahexaenoic acid + gamma-linolenic acid | diindolylmethane | nimesulide |
| docosahexaenoic acid + eicosapentaenoic acid | diindolylmethane | aspirin |
| docosahexaenoic acid + eicosapentaenoic acid | diindolylmethane | amoxiprin |
| docosahexaenoic acid + eicosapentaenoic acid | diindolylmethane | benorilate |
| docosahexaenoic acid + eicosapentaenoic acid | diindolylmethane | choline magnesium |
| | | salicylate |
| docosahexaenoic acid + eicosapentaenoic acid | diindolylmethane | diflunisal |
| docosahexaenoic acid + eicosapentaenoic acid | diindolylmethane | faislamine |
| docosahexaenoic acid + eicosapentaenoic acid | diindolylmethane | methylsalicylate |
| docosahexaenoic acid + eicosapentaenoic acid | diindolylmethane | salicylsalicylate |
| docosahexaenoic acid + eicosapentaenoic acid | diindolylmethane | diclofenac |
| docosahexaenoic acid + eicosapentaenoic acid | diindolylmethane | aceclofenac |
| docosahexaenoic acid + eicosapentaenoic acid | diindolylmethane | acemetacin |
| docosahexaenoic acid + eicosapentaenoic acid | diindolylmethane | bromfenac |
| docosahexaenoic acid + eicosapentaenoic acid | diindolylmethane | etodolac |
| docosahexaenoic acid + eicosapentaenoic acid | diindolylmethane | indometacin |
| docosahexaenoic acid + eicosapentaenoic acid | diindolylmethane | nabumetone |
| docosahexaenoic acid + eicosapentaenoic acid | diindolylmethane | sulindac |
| docosahexaenoic acid + eicosapentaenoic acid | diindolylmethane | tolmetin |
| docosahexaenoic acid + eicosapentaenoic acid | diindolylmethane | ibuprofen |
| docosahexaenoic acid + eicosapentaenoic acid | diindolylmethane | carprofen |
| docosahexaenoic acid + eicosapentaenoic acid | diindolylmethane | fenbufen |
| docosahexaenoic acid + eicosapentaenoic acid | diindolylmethane | fenoprofen |
| docosahexaenoic acid + eicosapentaenoic acid | diindolylmethane | flurbiprofen |
| docosahexaenoic acid + eicosapentaenoic acid | diindolylmethane | ketoprofen |
| docosahexaenoic acid + eicosapentaenoic acid | diindolylmethane | ketorolac |
| docosahexaenoic acid + eicosapentaenoic acid | diindolylmethane | loxoprofen |
| docosahexaenoic acid + eicosapentaenoic acid | diindolylmethane | naproxen |
| docosahexaenoic acid + eicosapentaenoic acid | diindolylmethane | tiaprofenicacid |
| docosahexaenoic acid + eicosapentaenoic acid | diindolylmethane | suprofen |
| docosahexaenoic acid + eicosapentaenoic acid | diindolylmethane | mefenamicacid |
| docosahexaenoic acid + eicosapentaenoic acid | diindolylmethane | meclofenamicacid |
| docosahexaenoic acid + eicosapentaenoic acid | diindolylmethane | phenylbutazone |
| docosahexaenoic acid + eicosapentaenoic acid | diindolylmethane | azapropazone |
| docosahexaenoic acid + eicosapentaenoic acid | diindolylmethane | metamizole |
| docosahexaenoic acid + eicosapentaenoic acid | diindolylmethane | oxyphenbutazone |
| docosahexaenoic acid + eicosapentaenoic acid | diindolylmethane | sulfinprazone |
| docosahexaenoic acid + eicosapentaenoic acid | diindolylmethane | piroxicam |
| docosahexaenoic acid + eicosapentaenoic acid | diindolylmethane | lornoxicam |
| docosahexaenoic acid + eicosapentaenoic acid | diindolylmethane | meloxicam |
| docosahexaenoic acid + eicosapentaenoic acid | diindolylmethane | tenoxicam |
| docosahexaenoic acid + eicosapentaenoic acid | diindolylmethane | celecoxib |
| docosahexaenoic acid + eicosapentaenoic acid | diindolylmethane | etoricoxib |
| docosahexaenoic acid + eicosapentaenoic acid | diindolylmethane | lumiracoxib |
| docosahexaenoic acid + eicosapentaenoic acid | diindolylmethane | parecoxib |
| docosahexaenoic acid + eicosapentaenoic acid | diindolylmethane | rofecoxib |
| docosahexaenoic acid + eicosapentaenoic acid | diindolylmethane | valdecoxib |
| docosahexaenoic acid + eicosapentaenoic acid | diindolylmethane | nimesulide |
| gamma-linolenic acid + eicosapentaenoic acid | diindolylmethane | aspirin |
| gamma-linolenic acid + eicosapentaenoic acid | diindolylmethane | amoxiprin |
| gamma-linolenic acid + eicosapentaenoic acid | diindolylmethane | benorilate |
| gamma-linolenic acid + eicosapentaenoic acid | diindolylmethane | choline magnesium |
| | | salicylate |
| gamma-linolenic acid + eicosapentaenoic acid | diindolylmethane | diflunisal |
| gamma-linolenic acid + eicosapentaenoic acid | diindolylmethane | faislamine |
| gamma-linolenic acid + eicosapentaenoic acid | diindolylmethane | methylsalicylate |
| gamma-linolenic acid + eicosapentaenoic acid | diindolylmethane | salicylsalicylate |
| gamma-linolenic acid + eicosapentaenoic acid | diindolylmethane | diclofenac |
| gamma-linolenic acid + eicosapentaenoic acid | diindolylmethane | aceclofenac |
| gamma-linolenic acid + eicosapentaenoic acid | diindolylmethane | acemetacin |
| gamma-linolenic acid + eicosapentaenoic acid | diindolylmethane | bromfenac |
| gamma-linolenic acid + eicosapentaenoic acid | diindolylmethane | etodolac |
| gamma-linolenic acid + eicosapentaenoic acid | diindolylmethane | indometacin |
| gamma-linolenic acid + eicosapentaenoic acid | diindolylmethane | nabumetone |
| gamma-linolenic acid + eicosapentaenoic acid | diindolylmethane | sulindac |
| gamma-linolenic acid + eicosapentaenoic acid | diindolylmethane | tolmetin |
| gamma-linolenic acid + eicosapentaenoic acid | diindolylmethane | ibuprofen |
| gamma-linolenic acid + eicosapentaenoic acid | diindolylmethane | carprofen |
| gamma-linolenic acid + eicosapentaenoic acid | diindolylmethane | fenbufen |
| gamma-linolenic acid + eicosapentaenoic acid | diindolylmethane | fenoprofen |
| gamma-linolenic acid + eicosapentaenoic acid | diindolylmethane | flurbiprofen |
| gamma-linolenic acid + eicosapentaenoic acid | diindolylmethane | ketoprofen |
| gamma-linolenic acid + eicosapentaenoic acid | diindolylmethane | ketorolac |
| gamma-linolenic acid + eicosapentaenoic acid | diindolylmethane | loxoprofen |
| gamma-linolenic acid + eicosapentaenoic acid | diindolylmethane | naproxen |
| gamma-linolenic acid + eicosapentaenoic acid | diindolylmethane | tiaprofenicacid |
| gamma-linolenic acid + eicosapentaenoic acid | diindolylmethane | suprofen |
| gamma-linolenic acid + eicosapentaenoic acid | diindolylmethane | mefenamicacid |
| gamma-linolenic acid + eicosapentaenoic acid | diindolylmethane | meclofenamicacid |
| gamma-linolenic acid + eicosapentaenoic acid | diindolylmethane | phenylbutazone |
| gamma-linolenic acid + eicosapentaenoic acid | diindolylmethane | azapropazone |
| gamma-linolenic acid + eicosapentaenoic acid | diindolylmethane | metamizole |
| gamma-linolenic acid + eicosapentaenoic acid | diindolylmethane | oxyphenbutazone |
| gamma-linolenic acid + eicosapentaenoic acid | diindolylmethane | sulfinprazone |
| gamma-linolenic acid + eicosapentaenoic acid | diindolylmethane | piroxicam |
| gamma-linolenic acid + eicosapentaenoic acid | diindolylmethane | lornoxicam |
| gamma-linolenic acid + eicosapentaenoic acid | diindolylmethane | meloxicam |
| gamma-linolenic acid + eicosapentaenoic acid | diindolylmethane | tenoxicam |
| gamma-linolenic acid + eicosapentaenoic acid | diindolylmethane | celecoxib |
| gamma-linolenic acid + eicosapentaenoic acid | diindolylmethane | etoricoxib |
| gamma-linolenic acid + eicosapentaenoic acid | diindolylmethane | lumiracoxib |
| gamma-linolenic acid + eicosapentaenoic acid | diindolylmethane | parecoxib |
| gamma-linolenic acid + eicosapentaenoic acid | diindolylmethane | rofecoxib |
| gamma-linolenic acid + eicosapentaenoic acid | diindolylmethane | valdecoxib |
| gamma-linolenic acid + eicosapentaenoic acid | diindolylmethane | nimesulide |
| alpha-linolenic acid + docosahexaenoic acid + | diindolylmethane | aspirin |
| gamma-linolenic acid | | |
| alpha-linolenic acid + docosahexaenoic acid + | diindolylmethane | amoxiprin |
| gamma-linolenic acid | | |
| alpha-linolenic acid + docosahexaenoic acid + | diindolylmethane | benorilate |
| gamma-linolenic acid | | |
| alpha-linolenic acid + docosahexaenoic acid + | diindolylmethane | choline magnesium |
| gamma-linolenic acid | | salicylate |
| alpha-linolenic acid + docosahexaenoic acid + | diindolylmethane | diflunisal |
| gamma-linolenic acid | | |
| alpha-linolenic acid + docosahexaenoic acid + | diindolylmethane | faislamine |
| gamma-linolenic acid | | |
| alpha-linolenic acid + docosahexaenoic acid + | diindolylmethane | methylsalicylate |
| gamma-linolenic acid | | |
| alpha-linolenic acid + docosahexaenoic acid + | diindolylmethane | salicylsalicylate |
| gamma-linolenic acid | | |
| alpha-linolenic acid + docosahexaenoic acid + | diindolylmethane | diclofenac |
| gamma-linolenic acid | | |
| alpha-linolenic acid + docosahexaenoic acid + | diindolylmethane | aceclofenac |
| gamma-linolenic acid | | |
| alpha-linolenic acid + docosahexaenoic acid + | diindolylmethane | acemetacin |
| gamma-linolenic acid | | |
| alpha-linolenic acid + docosahexaenoic acid + | diindolylmethane | bromfenac |
| gamma-linolenic acid | | |
| alpha-linolenic acid + docosahexaenoic acid + | diindolylmethane | etodolac |
| gamma-linolenic acid | | |
| alpha-linolenic acid + docosahexaenoic acid + | diindolylmethane | indometacin |
| gamma-linolenic acid | | |
| alpha-linolenic acid + docosahexaenoic acid + | diindolylmethane | nabumetone |
| gamma-linolenic acid | | |
| alpha-linolenic acid + docosahexaenoic acid + | diindolylmethane | sulindac |
| gamma-linolenic acid | | |
| alpha-linolenic acid + docosahexaenoic acid + | diindolylmethane | tolmetin |
| gamma-linolenic acid | | |
| alpha-linolenic acid + docosahexaenoic acid + | diindolylmethane | ibuprofen |
| gamma-linolenic acid | | |
| alpha-linolenic acid + docosahexaenoic acid + | diindolylmethane | carprofen |
| gamma-linolenic acid | | |
| alpha-linolenic acid + docosahexaenoic acid + | diindolylmethane | fenbufen |
| gamma-linolenic acid | | |
| alpha-linolenic acid + docosahexaenoic acid + | diindolylmethane | fenoprofen |
| gamma-linolenic acid | | |
| alpha-linolenic acid + docosahexaenoic acid + | diindolylmethane | flurbiprofen |
| gamma-linolenic acid | | |
| alpha-linolenic acid + docosahexaenoic acid + | diindolylmethane | ketoprofen |
| gamma-linolenic acid | | |
| alpha-linolenic acid + docosahexaenoic acid + | diindolylmethane | ketorolac |
| gamma-linolenic acid | | |
| alpha-linolenic acid + docosahexaenoic acid + | diindolylmethane | loxoprofen |
| gamma-linolenic acid | | |
| alpha-linolenic acid + docosahexaenoic acid + | diindolylmethane | naproxen |
| gamma-linolenic acid | | |
| alpha-linolenic acid + docosahexaenoic acid + | diindolylmethane | tiaprofenicacid |
| gamma-linolenic acid | | |
| alpha-linolenic acid + docosahexaenoic acid + | diindolylmethane | suprofen |
| gamma-linolenic acid | | |
| alpha-linolenic acid + docosahexaenoic acid + | diindolylmethane | mefenamicacid |
| gamma-linolenic acid | | |
| alpha-linolenic acid + docosahexaenoic acid + | diindolylmethane | meclofenamicacid |
| gamma-linolenic acid | | |
| alpha-linolenic acid + docosahexaenoic acid + | diindolylmethane | phenylbutazone |
| gamma-linolenic acid | | |
| alpha-linolenic acid + docosahexaenoic acid + | diindolylmethane | azapropazone |
| gamma-linolenic acid | | |
| alpha-linolenic acid + docosahexaenoic acid + | diindolylmethane | metamizole |
| gamma-linolenic acid | | |
| alpha-linolenic acid + docosahexaenoic acid + | diindolylmethane | oxyphenbutazone |
| gamma-linolenic acid | | |
| alpha-linolenic acid + docosahexaenoic acid + | diindolylmethane | sulfinprazone |
| gamma-linolenic acid | | |
| alpha-linolenic acid + docosahexaenoic acid + | diindolylmethane | piroxicam |
| gamma-linolenic acid | | |
| alpha-linolenic acid + docosahexaenoic acid + | diindolylmethane | lornoxicam |
| gamma-linolenic acid | | |
| alpha-linolenic acid + docosahexaenoic acid + | diindolylmethane | meloxicam |
| gamma-linolenic acid | | |
| alpha-linolenic acid + docosahexaenoic acid + | diindolylmethane | tenoxicam |
| gamma-linolenic acid | | |
| alpha-linolenic acid + docosahexaenoic acid + | diindolylmethane | celecoxib |
| gamma-linolenic acid | | |
| alpha-linolenic acid + docosahexaenoic acid + | diindolylmethane | etoricoxib |
| gamma-linolenic acid | | |
| alpha-linolenic acid + docosahexaenoic acid + | diindolylmethane | lumiracoxib |
| gamma-linolenic acid | | |
| alpha-linolenic acid + docosahexaenoic acid + | diindolylmethane | parecoxib |
| gamma-linolenic acid | | |
| alpha-linolenic acid + docosahexaenoic acid + | diindolylmethane | rofecoxib |
| gamma-linolenic acid | | |
| alpha-linolenic acid + docosahexaenoic acid + | diindolylmethane | valdecoxib |
| gamma-linolenic acid | | |
| alpha-linolenic acid + docosahexaenoic acid + | diindolylmethane | nimesulide |
| gamma-linolenic acid | | |
| alpha-linolenic acid + docosahexaenoic acid + | diindolylmethane | aspirin |
| eicosapentaenoic acid | | |
| alpha-linolenic acid + docosahexaenoic acid + | diindolylmethane | amoxiprin |
| eicosapentaenoic acid | | |
| alpha-linolenic acid + docosahexaenoic acid + | diindolylmethane | benorilate |
| eicosapentaenoic acid | | |
| alpha-linolenic acid + docosahexaenoic acid + | diindolylmethane | choline magnesium |
| eicosapentaenoic acid | | salicylate |
| alpha-linolenic acid + docosahexaenoic acid + | diindolylmethane | diflunisal |
| eicosapentaenoic acid | | |
| alpha-linolenic acid + docosahexaenoic acid + | diindolylmethane | faislamine |
| eicosapentaenoic acid | | |
| alpha-linolenic acid + docosahexaenoic acid + | diindolylmethane | methylsalicylate |
| eicosapentaenoic acid | | |
| alpha-linolenic acid + docosahexaenoic acid + | diindolylmethane | salicylsalicylate |
| eicosapentaenoic acid | | |
| alpha-linolenic acid + docosahexaenoic acid + | diindolylmethane | diclofenac |
| eicosapentaenoic acid | | |
| alpha-linolenic acid + docosahexaenoic acid + | diindolylmethane | aceclofenac |
| eicosapentaenoic acid | | |
| alpha-linolenic acid + docosahexaenoic acid + | diindolylmethane | acemetacin |
| eicosapentaenoic acid | | |
| alpha-linolenic acid + docosahexaenoic acid + | diindolylmethane | bromfenac |
| eicosapentaenoic acid | | |
| alpha-linolenic acid + docosahexaenoic acid + | diindolylmethane | etodolac |
| eicosapentaenoic acid | | |
| alpha-linolenic acid + docosahexaenoic acid + | diindolylmethane | indometacin |
| eicosapentaenoic acid | | |
| alpha-linolenic acid + docosahexaenoic acid + | diindolylmethane | nabumetone |
| eicosapentaenoic acid | | |
| alpha-linolenic acid + docosahexaenoic acid + | diindolylmethane | sulindac |
| eicosapentaenoic acid | | |
| alpha-linolenic acid + docosahexaenoic acid + | diindolylmethane | tolmetin |
| eicosapentaenoic acid | | |
| alpha-linolenic acid + docosahexaenoic acid + | diindolylmethane | ibuprofen |
| eicosapentaenoic acid | | |
| alpha-linolenic acid + docosahexaenoic acid + | diindolylmethane | carprofen |
| eicosapentaenoic acid | | |
| alpha-linolenic acid + docosahexaenoic acid + | diindolylmethane | fenbufen |
| eicosapentaenoic acid | | |
| alpha-linolenic acid + docosahexaenoic acid + | diindolylmethane | fenoprofen |
| eicosapentaenoic acid | | |
| alpha-linolenic acid + docosahexaenoic acid + | diindolylmethane | flurbiprofen |
| eicosapentaenoic acid | | |
| alpha-linolenic acid + docosahexaenoic acid + | diindolylmethane | ketoprofen |
| eicosapentaenoic acid | | |
| alpha-linolenic acid + docosahexaenoic acid + | diindolylmethane | ketorolac |
| eicosapentaenoic acid | | |
| alpha-linolenic acid + docosahexaenoic acid + | diindolylmethane | loxoprofen |
| eicosapentaenoic acid | | |
| alpha-linolenic acid + docosahexaenoic acid + | diindolylmethane | naproxen |
| eicosapentaenoic acid | | |
| alpha-linolenic acid + docosahexaenoic acid + | diindolylmethane | tiaprofenicacid |
| eicosapentaenoic acid | | |
| alpha-linolenic acid + docosahexaenoic acid + | diindolylmethane | suprofen |
| eicosapentaenoic acid | | |
| alpha-linolenic acid + docosahexaenoic acid + | diindolylmethane | mefenamicacid |
| eicosapentaenoic acid | | |
| alpha-linolenic acid + docosahexaenoic acid + | diindolylmethane | meclofenamicacid |
| eicosapentaenoic acid | | |
| alpha-linolenic acid + docosahexaenoic acid + | diindolylmethane | phenylbutazone |
| eicosapentaenoic acid | | |
| alpha-linolenic acid + docosahexaenoic acid + | diindolylmethane | azapropazone |
| eicosapentaenoic acid | | |
| alpha-linolenic acid + docosahexaenoic acid + | diindolylmethane | metamizole |
| eicosapentaenoic acid | | |
| alpha-linolenic acid + docosahexaenoic acid + | diindolylmethane | oxyphenbutazone |
| eicosapentaenoic acid | | |
| alpha-linolenic acid + docosahexaenoic acid + | diindolylmethane | sulfinprazone |
| eicosapentaenoic acid | | |
| alpha-linolenic acid + docosahexaenoic acid + | diindolylmethane | piroxicam |
| eicosapentaenoic acid | | |
| alpha-linolenic acid + docosahexaenoic acid + | diindolylmethane | lornoxicam |
| eicosapentaenoic acid | | |
| alpha-linolenic acid + docosahexaenoic acid + | diindolylmethane | meloxicam |
| eicosapentaenoic acid | | |
| alpha-linolenic acid + docosahexaenoic acid + | diindolylmethane | tenoxicam |
| eicosapentaenoic acid | | |
| alpha-linolenic acid + docosahexaenoic acid + | diindolylmethane | celecoxib |
| eicosapentaenoic acid | | |
| alpha-linolenic acid + docosahexaenoic acid + | diindolylmethane | etoricoxib |
| eicosapentaenoic acid | | |
| alpha-linolenic acid + docosahexaenoic acid + | diindolylmethane | lumiracoxib |
| eicosapentaenoic acid | | |
| alpha-linolenic acid + docosahexaenoic acid + | diindolylmethane | parecoxib |
| eicosapentaenoic acid | | |
| alpha-linolenic acid + docosahexaenoic acid + | diindolylmethane | rofecoxib |
| eicosapentaenoic acid | | |
| alpha-linolenic acid + docosahexaenoic acid + | diindolylmethane | valdecoxib |
| eicosapentaenoic acid | | |
| alpha-linolenic acid + docosahexaenoic acid + | diindolylmethane | nimesulide |
| eicosapentaenoic acid | | |
| docosahexaenoic acid + gamma-linolenic acid + | diindolylmethane | aspirin |
| eicosapentaenoic acid | | |
| docosahexaenoic acid + gamma-linolenic acid + | diindolylmethane | amoxiprin |
| eicosapentaenoic acid | | |
| docosahexaenoic acid + gamma-linolenic acid + | diindolylmethane | benorilate |
| eicosapentaenoic acid | | |
| docosahexaenoic acid + gamma-linolenic acid + | diindolylmethane | choline magnesium |
| eicosapentaenoic acid | | salicylate |
| docosahexaenoic acid + gamma-linolenic acid + | diindolylmethane | diflunisal |
| eicosapentaenoic acid | | |
| docosahexaenoic acid + gamma-linolenic acid + | diindolylmethane | faislamine |
| eicosapentaenoic acid | | |
| docosahexaenoic acid + gamma-linolenic acid + | diindolylmethane | methylsalicylate |
| eicosapentaenoic acid | | |
| docosahexaenoic acid + gamma-linolenic acid + | diindolylmethane | salicylsalicylate |
| eicosapentaenoic acid | | |
| docosahexaenoic acid + gamma-linolenic acid + | diindolylmethane | diclofenac |
| eicosapentaenoic acid | | |
| docosahexaenoic acid + gamma-linolenic acid + | diindolylmethane | aceclofenac |
| eicosapentaenoic acid | | |
| docosahexaenoic acid + gamma-linolenic acid + | diindolylmethane | acemetacin |
| eicosapentaenoic acid | | |
| docosahexaenoic acid + gamma-linolenic acid + | diindolylmethane | bromfenac |
| eicosapentaenoic acid | | |
| docosahexaenoic acid + gamma-linolenic acid + | diindolylmethane | etodolac |
| eicosapentaenoic acid | | |
| docosahexaenoic acid + gamma-linolenic acid + | diindolylmethane | indometacin |
| eicosapentaenoic acid | | |
| docosahexaenoic acid + gamma-linolenic acid + | diindolylmethane | nabumetone |
| eicosapentaenoic acid | | |
| docosahexaenoic acid + gamma-linolenic acid + | diindolylmethane | sulindac |
| eicosapentaenoic acid | | |
| docosahexaenoic acid + gamma-linolenic acid + | diindolylmethane | tolmetin |
| eicosapentaenoic acid | | |
| docosahexaenoic acid + gamma-linolenic acid + | diindolylmethane | ibuprofen |
| eicosapentaenoic acid | | |
| docosahexaenoic acid + gamma-linolenic acid + | diindolylmethane | carprofen |
| eicosapentaenoic acid | | |
| docosahexaenoic acid + gamma-linolenic acid + | diindolylmethane | fenbufen |
| eicosapentaenoic acid | | |
| docosahexaenoic acid + gamma-linolenic acid + | diindolylmethane | fenoprofen |
| eicosapentaenoic acid | | |
| docosahexaenoic acid + gamma-linolenic acid + | diindolylmethane | flurbiprofen |
| eicosapentaenoic acid | | |
| docosahexaenoic acid + gamma-linolenic acid + | diindolylmethane | ketoprofen |
| eicosapentaenoic acid | | |
| docosahexaenoic acid + gamma-linolenic acid + | diindolylmethane | ketorolac |
| eicosapentaenoic acid | | |
| docosahexaenoic acid + gamma-linolenic acid + | diindolylmethane | loxoprofen |
| eicosapentaenoic acid | | |
| docosahexaenoic acid + gamma-linolenic acid + | diindolylmethane | naproxen |
| eicosapentaenoic acid | | |
| docosahexaenoic acid + gamma-linolenic acid + | diindolylmethane | tiaprofenicacid |
| eicosapentaenoic acid | | |
| docosahexaenoic acid + gamma-linolenic acid + | diindolylmethane | suprofen |
| eicosapentaenoic acid | | |
| docosahexaenoic acid + gamma-linolenic acid + | diindolylmethane | mefenamicacid |
| eicosapentaenoic acid | | |
| docosahexaenoic acid + gamma-linolenic acid + | diindolylmethane | meclofenamicacid |
| eicosapentaenoic acid | | |
| docosahexaenoic acid + gamma-linolenic acid + | diindolylmethane | phenylbutazone |
| eicosapentaenoic acid | | |
| docosahexaenoic acid + gamma-linolenic acid + | diindolylmethane | azapropazone |
| eicosapentaenoic acid | | |
| docosahexaenoic acid + gamma-linolenic acid + | diindolylmethane | metamizole |
| eicosapentaenoic acid | | |
| docosahexaenoic acid + gamma-linolenic acid + | diindolylmethane | oxyphenbutazone |
| eicosapentaenoic acid | | |
| docosahexaenoic acid + gamma-linolenic acid + | diindolylmethane | sulfinprazone |
| eicosapentaenoic acid | | |
| docosahexaenoic acid + gamma-linolenic acid + | diindolylmethane | piroxicam |
| eicosapentaenoic acid | | |
| docosahexaenoic acid + gamma-linolenic acid + | diindolylmethane | lornoxicam |
| eicosapentaenoic acid | | |
| docosahexaenoic acid + gamma-linolenic acid + | diindolylmethane | meloxicam |
| eicosapentaenoic acid | | |
| docosahexaenoic acid + gamma-linolenic acid + | diindolylmethane | tenoxicam |
| eicosapentaenoic acid | | |
| docosahexaenoic acid + gamma-linolenic acid + | diindolylmethane | celecoxib |
| eicosapentaenoic acid | | |
| docosahexaenoic acid + gamma-linolenic acid + | diindolylmethane | etoricoxib |
| eicosapentaenoic acid | | |
| docosahexaenoic acid + gamma-linolenic acid + | diindolylmethane | lumiracoxib |
| eicosapentaenoic acid | | |
| docosahexaenoic acid + gamma-linolenic acid + | diindolylmethane | parecoxib |
| eicosapentaenoic acid | | |
| docosahexaenoic acid + gamma-linolenic acid + | diindolylmethane | rofecoxib |
| eicosapentaenoic acid | | |
| docosahexaenoic acid + gamma-linolenic acid + | diindolylmethane | valdecoxib |
| eicosapentaenoic acid | | |
| docosahexaenoic acid + gamma-linolenic acid + | diindolylmethane | nimesulide |
| eicosapentaenoic acid | | |
| alpha-linolenic acid + gamma-linolenic acid + | diindolylmethane | aspirin |
| eicosapentaenoic acid | | |
| alpha-linolenic acid + gamma-linolenic acid + | diindolylmethane | amoxiprin |
| eicosapentaenoic acid | | |
| alpha-linolenic acid + gamma-linolenic acid + | diindolylmethane | benorilate |
| eicosapentaenoic acid | | |
| alpha-linolenic acid + gamma-linolenic acid + | diindolylmethane | choline magnesium |
| eicosapentaenoic acid | | salicylate |
| alpha-linolenic acid + gamma-linolenic acid + | diindolylmethane | diflunisal |
| eicosapentaenoic acid | | |
| alpha-linolenic acid + gamma-linolenic acid + | diindolylmethane | faislamine |
| eicosapentaenoic acid | | |
| alpha-linolenic acid + gamma-linolenic acid + | diindolylmethane | methylsalicylate |
| eicosapentaenoic acid | | |
| alpha-linolenic acid + gamma-linolenic acid + | diindolylmethane | salicylsalicylate |
| eicosapentaenoic acid | | |
| alpha-linolenic acid + gamma-linolenic acid + | diindolylmethane | diclofenac |
| eicosapentaenoic acid | | |
| alpha-linolenic acid + gamma-linolenic acid + | diindolylmethane | aceclofenac |
| eicosapentaenoic acid | | |
| alpha-linolenic acid + gamma-linolenic acid + | diindolylmethane | acemetacin |
| eicosapentaenoic acid | | |
| alpha-linolenic acid + gamma-linolenic acid + | diindolylmethane | bromfenac |
| eicosapentaenoic acid | | |
| alpha-linolenic acid + gamma-linolenic acid + | diindolylmethane | etodolac |
| eicosapentaenoic acid | | |
| alpha-linolenic acid + gamma-linolenic acid + | diindolylmethane | indometacin |
| eicosapentaenoic acid | | |
| alpha-linolenic acid + gamma-linolenic acid + | diindolylmethane | nabumetone |
| eicosapentaenoic acid | | |
| alpha-linolenic acid + gamma-linolenic acid + | diindolylmethane | sulindac |
| eicosapentaenoic acid | | |
| alpha-linolenic acid + gamma-linolenic acid + | diindolylmethane | tolmetin |
| eicosapentaenoic acid | | |
| alpha-linolenic acid + gamma-linolenic acid + | diindolylmethane | ibuprofen |
| eicosapentaenoic acid | | |
| alpha-linolenic acid + gamma-linolenic acid + | diindolylmethane | carprofen |
| eicosapentaenoic acid | | |
| alpha-linolenic acid + gamma-linolenic acid + | diindolylmethane | fenbufen |
| eicosapentaenoic acid | | |
| alpha-linolenic acid + gamma-linolenic acid + | diindolylmethane | fenoprofen |
| eicosapentaenoic acid | | |
| alpha-linolenic acid + gamma-linolenic acid + | diindolylmethane | flurbiprofen |
| eicosapentaenoic acid | | |
| alpha-linolenic acid + gamma-linolenic acid + | diindolylmethane | ketoprofen |
| eicosapentaenoic acid | | |
| alpha-linolenic acid + gamma-linolenic acid + | diindolylmethane | ketorolac |
| eicosapentaenoic acid | | |
| alpha-linolenic acid + gamma-linolenic acid + | diindolylmethane | loxoprofen |
| eicosapentaenoic acid | | |
| alpha-linolenic acid + gamma-linolenic acid + | diindolylmethane | naproxen |
| eicosapentaenoic acid | | |
| alpha-linolenic acid + gamma-linolenic acid + | diindolylmethane | tiaprofenicacid |
| eicosapentaenoic acid | | |
| alpha-linolenic acid + gamma-linolenic acid + | diindolylmethane | suprofen |
| eicosapentaenoic acid | | |
| alpha-linolenic acid + gamma-linolenic acid + | diindolylmethane | mefenamicacid |
| eicosapentaenoic acid | | |
| alpha-linolenic acid + gamma-linolenic acid + | diindolylmethane | meclofenamicacid |
| eicosapentaenoic acid | | |
| alpha-linolenic acid + gamma-linolenic acid + | diindolylmethane | phenylbutazone |
| eicosapentaenoic acid | | |
| alpha-linolenic acid + gamma-linolenic acid + | diindolylmethane | azapropazone |
| eicosapentaenoic acid | | |
| alpha-linolenic acid + gamma-linolenic acid + | diindolylmethane | metamizole |
| eicosapentaenoic acid | | |
| alpha-linolenic acid + gamma-linolenic acid + | diindolylmethane | oxyphenbutazone |
| eicosapentaenoic acid | | |
| alpha-linolenic acid + gamma-linolenic acid + | diindolylmethane | sulfinprazone |
| eicosapentaenoic acid | | |
| alpha-linolenic acid + gamma-linolenic acid + | diindolylmethane | piroxicam |
| eicosapentaenoic acid | | |
| alpha-linolenic acid + gamma-linolenic acid + | diindolylmethane | lornoxicam |
| eicosapentaenoic acid | | |
| alpha-linolenic acid + gamma-linolenic acid + | diindolylmethane | meloxicam |
| eicosapentaenoic acid | | |
| alpha-linolenic acid + gamma-linolenic acid + | diindolylmethane | tenoxicam |
| eicosapentaenoic acid | | |
| alpha-linolenic acid + gamma-linolenic acid + | diindolylmethane | celecoxib |
| eicosapentaenoic acid | | |
| alpha-linolenic acid + gamma-linolenic acid + | diindolylmethane | etoricoxib |
| eicosapentaenoic acid | | |
| alpha-linolenic acid + gamma-linolenic acid + | diindolylmethane | lumiracoxib |
| eicosapentaenoic acid | | |
| alpha-linolenic acid + gamma-linolenic acid + | diindolylmethane | parecoxib |
| eicosapentaenoic acid | | |
| alpha-linolenic acid + gamma-linolenic acid + | diindolylmethane | rofecoxib |
| eicosapentaenoic acid | | |
| alpha-linolenic acid + gamma-linolenic acid + | diindolylmethane | valdecoxib |
| eicosapentaenoic acid | | |
| alpha-linolenic acid + gamma-linolenic acid + | diindolylmethane | nimesulide |
| eicosapentaenoic acid | | |
| alpha-linolenic acid + docosahexaenoic acid + | diindolylmethane | aspirin |
| gamma-linolenic acid + eicosapentaenoic acid | | |
| alpha-linolenic acid + docosahexaenoic acid + | diindolylmethane | amoxiprin |
| gamma-linolenic acid + eicosapentaenoic acid | | |
| alpha-linolenic acid + docosahexaenoic acid + | diindolylmethane | benorilate |
| gamma-linolenic acid + eicosapentaenoic acid | | |
| alpha-linolenic acid + docosahexaenoic acid + | diindolylmethane | choline magnesium |
| gamma-linolenic acid + eicosapentaenoic acid | | salicylate |
| alpha-linolenic acid + docosahexaenoic acid + | diindolylmethane | diflunisal |
| gamma-linolenic acid + eicosapentaenoic acid | | |
| alpha-linolenic acid + docosahexaenoic acid + | diindolylmethane | faislamine |
| gamma-linolenic acid + eicosapentaenoic acid | | |
| alpha-linolenic acid + docosahexaenoic acid + | diindolylmethane | methylsalicylate |
| gamma-linolenic acid + eicosapentaenoic acid | | |
| alpha-linolenic acid + docosahexaenoic acid + | diindolylmethane | salicylsalicylate |
| gamma-linolenic acid + eicosapentaenoic acid | | |
| alpha-linolenic acid + docosahexaenoic acid + | diindolylmethane | diclofenac |
| gamma-linolenic acid + eicosapentaenoic acid | | |
| alpha-linolenic acid + docosahexaenoic acid + | diindolylmethane | aceclofenac |
| gamma-linolenic acid + eicosapentaenoic acid | | |
| alpha-linolenic acid + docosahexaenoic acid + | diindolylmethane | acemetacin |
| gamma-linolenic acid + eicosapentaenoic acid | | |
| alpha-linolenic acid + docosahexaenoic acid + | diindolylmethane | bromfenac |
| gamma-linolenic acid + eicosapentaenoic acid | | |
| alpha-linolenic acid + docosahexaenoic acid + | diindolylmethane | etodolac |
| gamma-linolenic acid + eicosapentaenoic acid | | |
| alpha-linolenic acid + docosahexaenoic acid + | diindolylmethane | indometacin |
| gamma-linolenic acid + eicosapentaenoic acid | | |
| alpha-linolenic acid + docosahexaenoic acid + | diindolylmethane | nabumetone |
| gamma-linolenic acid + eicosapentaenoic acid | | |
| alpha-linolenic acid + docosahexaenoic acid + | diindolylmethane | sulindac |
| gamma-linolenic acid + eicosapentaenoic acid | | |
| alpha-linolenic acid + docosahexaenoic acid + | diindolylmethane | tolmetin |
| gamma-linolenic acid + eicosapentaenoic acid | | |
| alpha-linolenic acid + docosahexaenoic acid + | diindolylmethane | ibuprofen |
| gamma-linolenic acid + eicosapentaenoic acid | | |
| alpha-linolenic acid + docosahexaenoic acid + | diindolylmethane | carprofen |
| gamma-linolenic acid + eicosapentaenoic acid | | |
| alpha-linolenic acid + docosahexaenoic acid + | diindolylmethane | fenbufen |
| gamma-linolenic acid + eicosapentaenoic acid | | |
| alpha-linolenic acid + docosahexaenoic acid + | diindolylmethane | fenoprofen |
| gamma-linolenic acid + eicosapentaenoic acid | | |
| alpha-linolenic acid + docosahexaenoic acid + | diindolylmethane | flurbiprofen |
| gamma-linolenic acid + eicosapentaenoic acid | | |
| alpha-linolenic acid + docosahexaenoic acid + | diindolylmethane | ketoprofen |
| gamma-linolenic acid + eicosapentaenoic acid | | |
| alpha-linolenic acid + docosahexaenoic acid + | diindolylmethane | ketorolac |
| gamma-linolenic acid + eicosapentaenoic acid | | |
| alpha-linolenic acid + docosahexaenoic acid + | diindolylmethane | loxoprofen |
| gamma-linolenic acid + eicosapentaenoic acid | | |
| alpha-linolenic acid + docosahexaenoic acid + | diindolylmethane | naproxen |
| gamma-linolenic acid + eicosapentaenoic acid | | |
| alpha-linolenic acid + docosahexaenoic acid + | diindolylmethane | tiaprofenicacid |
| gamma-linolenic acid + eicosapentaenoic acid | | |
| alpha-linolenic acid + docosahexaenoic acid + | diindolylmethane | suprofen |
| gamma-linolenic acid + eicosapentaenoic acid | | |
| alpha-linolenic acid + docosahexaenoic acid + | diindolylmethane | mefenamicacid |
| gamma-linolenic acid + eicosapentaenoic acid | | |
| alpha-linolenic acid + docosahexaenoic acid + | diindolylmethane | meclofenamicacid |
| gamma-linolenic acid + eicosapentaenoic acid | | |
| alpha-linolenic acid + docosahexaenoic acid + | diindolylmethane | phenylbutazone |
| gamma-linolenic acid + eicosapentaenoic acid | | |
| alpha-linolenic acid + docosahexaenoic acid + | diindolylmethane | azapropazone |
| gamma-linolenic acid + eicosapentaenoic acid | | |
| alpha-linolenic acid + docosahexaenoic acid + | diindolylmethane | metamizole |
| gamma-linolenic acid + eicosapentaenoic acid | | |
| alpha-linolenic acid + docosahexaenoic acid + | diindolylmethane | oxyphenbutazone |
| gamma-linolenic acid + eicosapentaenoic acid | | |
| alpha-linolenic acid + docosahexaenoic acid + | diindolylmethane | sulfinprazone |
| gamma-linolenic acid + eicosapentaenoic acid | | |
| alpha-linolenic acid + docosahexaenoic acid + | diindolylmethane | piroxicam |
| gamma-linolenic acid + eicosapentaenoic acid | | |
| alpha-linolenic acid + docosahexaenoic acid + | diindolylmethane | lornoxicam |
| gamma-linolenic acid + eicosapentaenoic acid | | |
| alpha-linolenic acid + docosahexaenoic acid + | diindolylmethane | meloxicam |
| gamma-linolenic acid + eicosapentaenoic acid | | |
| alpha-linolenic acid + docosahexaenoic acid + | diindolylmethane | tenoxicam |
| gamma-linolenic acid + eicosapentaenoic acid | | |
| alpha-linolenic acid + docosahexaenoic acid + | diindolylmethane | celecoxib |
| gamma-linolenic acid + eicosapentaenoic acid | | |
| alpha-linolenic acid + docosahexaenoic acid + | diindolylmethane | etoricoxib |
| gamma-linolenic acid + eicosapentaenoic acid | | |
| alpha-linolenic acid + docosahexaenoic acid + | diindolylmethane | lumiracoxib |
| gamma-linolenic acid + eicosapentaenoic acid | | |
| alpha-linolenic acid + docosahexaenoic acid + | diindolylmethane | parecoxib |
| gamma-linolenic acid + eicosapentaenoic acid | | |
| alpha-linolenic acid + docosahexaenoic acid + | diindolylmethane | rofecoxib |
| gamma-linolenic acid + eicosapentaenoic acid | | |
| alpha-linolenic acid + docosahexaenoic acid + | diindolylmethane | valdecoxib |
| gamma-linolenic acid + eicosapentaenoic acid | | |
| alpha-linolenic acid + docosahexaenoic acid + | diindolylmethane | nimesulide |
| gamma-linolenic acid + eicosapentaenoic acid | | |

In an alternative exemplary embodiment, folic acid may be combined with any of the combinations delineated in Table B.

An additional exemplary combination comprises at least one polyunsaturated fatty acid, diindolylmethane, and a hormone. Non-limiting examples of this combination are delineated in Table C.

TABLE C

| First Agent | Second Agent | Third Agent |
|---|---|---|
| alpha-linolenic acid | diindolylmethane | estrogen |
| alpha-linolenic acid | diindolylmethane | conjugated estrogens |
| alpha-linolenic acid | diindolylmethane | dienestrol |
| alpha-linolenic acid | diindolylmethane | estrone |
| alpha-linolenic acid | diindolylmethane | esterified estrogens |
| alpha-linolenic acid | diindolylmethane | estradiol |
| alpha-linolenic acid | diindolylmethane | estriol |
| alpha-linolenic acid | diindolylmethane | estropipate |
| alpha-linolenic acid | diindolylmethane | ethinyl estradiol |
| alpha-linolenic acid | diindolylmethane | testosterone |
| alpha-linolenic acid | diindolylmethane | dehydroepiandrosterone |
| alpha-linolenic acid | diindolylmethane | androstenedione |
| alpha-linolenic acid | diindolylmethane | androstenediol |
| alpha-linolenic acid | diindolylmethane | androsterone |
| alpha-linolenic acid | diindolylmethane | dihydrotestosterone |
| docosahexaenoic acid | diindolylmethane | estrogen |
| docosahexaenoic acid | diindolylmethane | conjugated estrogens |
| docosahexaenoic acid | diindolylmethane | dienestrol |
| docosahexaenoic acid | diindolylmethane | estrone |
| docosahexaenoic acid | diindolylmethane | esterified estrogens |
| docosahexaenoic acid | diindolylmethane | estradiol |
| docosahexaenoic acid | diindolylmethane | estriol |
| docosahexaenoic acid | diindolylmethane | estropipate |
| docosahexaenoic acid | diindolylmethane | ethinyl estradiol |
| docosahexaenoic acid | diindolylmethane | testosterone |
| docosahexaenoic acid | diindolylmethane | dehydroepiandrosterone |
| docosahexaenoic acid | diindolylmethane | androstenedione |
| docosahexaenoic acid | diindolylmethane | androstenediol |
| docosahexaenoic acid | diindolylmethane | androsterone |
| docosahexaenoic acid | diindolylmethane | dihydrotestosterone |
| gamma-linolenic acid | diindolylmethane | estrogen |
| gamma-linolenic acid | diindolylmethane | conjugated estrogens |
| gamma-linolenic acid | diindolylmethane | dienestrol |
| gamma-linolenic acid | diindolylmethane | estrone |
| gamma-linolenic acid | diindolylmethane | esterified estrogens |
| gamma-linolenic acid | diindolylmethane | estradiol |
| gamma-linolenic acid | diindolylmethane | estriol |
| gamma-linolenic acid | diindolylmethane | estropipate |
| gamma-linolenic acid | diindolylmethane | ethinyl estradiol |
| gamma-linolenic acid | diindolylmethane | testosterone |
| gamma-linolenic acid | diindolylmethane | dehydroepiandrosterone |
| gamma-linolenic acid | diindolylmethane | androstenedione |
| gamma-linolenic acid | diindolylmethane | androstenediol |
| gamma-linolenic acid | diindolylmethane | androsterone |
| gamma-linolenic acid | diindolylmethane | dihydrotestosterone |
| eicosapentaenoic acid | diindolylmethane | estrogen |
| eicosapentaenoic acid | diindolylmethane | conjugated estrogens |
| eicosapentaenoic acid | diindolylmethane | dienestrol |
| eicosapentaenoic acid | diindolylmethane | estrone |
| eicosapentaenoic acid | diindolylmethane | esterified estrogens |
| eicosapentaenoic acid | diindolylmethane | estradiol |
| eicosapentaenoic acid | diindolylmethane | estriol |
| eicosapentaenoic acid | diindolylmethane | estropipate |
| eicosapentaenoic acid | diindolylmethane | ethinyl estradiol |
| eicosapentaenoic acid | diindolylmethane | testosterone |
| eicosapentaenoic acid | diindolylmethane | dehydroepiandrosterone |
| eicosapentaenoic acid | diindolylmethane | androstenedione |
| eicosapentaenoic acid | diindolylmethane | androstenediol |
| eicosapentaenoic acid | diindolylmethane | androsterone |
| eicosapentaenoic acid | diindolylmethane | dihydrotestosterone |
| alpha-linolenic acid + docosahexaenoic acid | diindolylmethane | estrogen |
| alpha-linolenic acid + docosahexaenoic acid | diindolylmethane | conjugated estrogens |
| alpha-linolenic acid + docosahexaenoic acid | diindolylmethane | dienestrol |
| alpha-linolenic acid + docosahexaenoic acid | diindolylmethane | estrone |
| alpha-linolenic acid + docosahexaenoic acid | diindolylmethane | esterified estrogens |
| alpha-linolenic acid + docosahexaenoic acid | diindolylmethane | estradiol |
| alpha-linolenic acid + docosahexaenoic acid | diindolylmethane | estriol |
| alpha-linolenic acid + docosahexaenoic acid | diindolylmethane | estropipate |
| alpha-linolenic acid + docosahexaenoic acid | diindolylmethane | ethinyl estradiol |
| alpha-linolenic acid + docosahexaenoic acid | diindolylmethane | testosterone |
| alpha-linolenic acid + docosahexaenoic acid | diindolylmethane | dehydroepiandrosterone |
| alpha-linolenic acid + docosahexaenoic acid | diindolylmethane | androstenedione |
| alpha-linolenic acid + docosahexaenoic acid | diindolylmethane | androstenediol |
| alpha-linolenic acid + docosahexaenoic acid | diindolylmethane | androsterone |
| alpha-linolenic acid + docosahexaenoic acid | diindolylmethane | dihydrotestosterone |
| alpha-linolenic acid + gamma-linolenic acid | diindolylmethane | estrogen |
| alpha-linolenic acid + gamma-linolenic acid | diindolylmethane | conjugated estrogens |
| alpha-linolenic acid + gamma-linolenic acid | diindolylmethane | dienestrol |
| alpha-linolenic acid + gamma-linolenic acid | diindolylmethane | estrone |
| alpha-linolenic acid + gamma-linolenic acid | diindolylmethane | esterified estrogens |
| alpha-linolenic acid + gamma-linolenic acid | diindolylmethane | estradiol |
| alpha-linolenic acid + gamma-linolenic acid | diindolylmethane | estriol |
| alpha-linolenic acid + gamma-linolenic acid | diindolylmethane | estropipate |
| alpha-linolenic acid + gamma-linolenic acid | diindolylmethane | ethinyl estradiol |
| alpha-linolenic acid + gamma-linolenic acid | diindolylmethane | testosterone |
| alpha-linolenic acid + gamma-linolenic acid | diindolylmethane | dehydroepiandrosterone |
| alpha-linolenic acid + gamma-linolenic acid | diindolylmethane | androstenedione |
| alpha-linolenic acid + gamma-linolenic acid | diindolylmethane | androstenediol |
| alpha-linolenic acid + gamma-linolenic acid | diindolylmethane | androsterone |
| alpha-linolenic acid + gamma-linolenic acid | diindolylmethane | dihydrotestosterone |
| alpha-linolenic acid + eicosapentaenoic acid | diindolylmethane | estrogen |
| alpha-linolenic acid + eicosapentaenoic acid | diindolylmethane | conjugated estrogens |
| alpha-linolenic acid + eicosapentaenoic acid | diindolylmethane | dienestrol |
| alpha-linolenic acid + eicosapentaenoic acid | diindolylmethane | estrone |
| alpha-linolenic acid + eicosapentaenoic acid | diindolylmethane | esterified estrogens |
| alpha-linolenic acid + eicosapentaenoic acid | diindolylmethane | estradiol |
| alpha-linolenic acid + eicosapentaenoic acid | diindolylmethane | estriol |
| alpha-linolenic acid + eicosapentaenoic acid | diindolylmethane | estropipate |
| alpha-linolenic acid + eicosapentaenoic acid | diindolylmethane | ethinyl estradiol |
| alpha-linolenic acid + eicosapentaenoic acid | diindolylmethane | testosterone |
| alpha-linolenic acid + eicosapentaenoic acid | diindolylmethane | dehydroepiandrosterone |
| alpha-linolenic acid + eicosapentaenoic acid | diindolylmethane | androstenedione |
| alpha-linolenic acid + eicosapentaenoic acid | diindolylmethane | androstenediol |
| alpha-linolenic acid + eicosapentaenoic acid | diindolylmethane | androsterone |
| alpha-linolenic acid + eicosapentaenoic acid | diindolylmethane | dihydrotestosterone |
| docosahexaenoic acid + gamma-linolenic acid | diindolylmethane | estrogen |
| docosahexaenoic acid + gamma-linolenic acid | diindolylmethane | conjugated estrogens |
| docosahexaenoic acid + gamma-linolenic acid | diindolylmethane | dienestrol |
| docosahexaenoic acid + gamma-linolenic acid | diindolylmethane | estrone |
| docosahexaenoic acid + gamma-linolenic acid | diindolylmethane | esterified estrogens |
| docosahexaenoic acid + gamma-linolenic acid | diindolylmethane | estradiol |
| docosahexaenoic acid + gamma-linolenic acid | diindolylmethane | estriol |
| docosahexaenoic acid + gamma-linolenic acid | diindolylmethane | estropipate |
| docosahexaenoic acid + gamma-linolenic acid | diindolylmethane | ethinyl estradiol |
| docosahexaenoic acid + gamma-linolenic acid | diindolylmethane | testosterone |
| docosahexaenoic acid + gamma-linolenic acid | diindolylmethane | dehydroepiandrosterone |
| docosahexaenoic acid + gamma-linolenic acid | diindolylmethane | androstenedione |
| docosahexaenoic acid + gamma-linolenic acid | diindolylmethane | androstenediol |
| docosahexaenoic acid + gamma-linolenic acid | diindolylmethane | androsterone |
| docosahexaenoic acid + gamma-linolenic acid | diindolylmethane | dihydrotestosterone |
| docosahexaenoic acid + eicosapentaenoic acid | diindolylmethane | estrogen |
| docosahexaenoic acid + eicosapentaenoic acid | diindolylmethane | conjugated estrogens |
| docosahexaenoic acid + eicosapentaenoic acid | diindolylmethane | dienestrol |
| docosahexaenoic acid + eicosapentaenoic acid | diindolylmethane | estrone |
| docosahexaenoic acid + eicosapentaenoic acid | diindolylmethane | esterified estrogens |
| docosahexaenoic acid + eicosapentaenoic acid | diindolylmethane | estradiol |
| docosahexaenoic acid + eicosapentaenoic acid | diindolylmethane | estriol |
| docosahexaenoic acid + eicosapentaenoic acid | diindolylmethane | estropipate |
| docosahexaenoic acid + eicosapentaenoic acid | diindolylmethane | ethinyl estradiol |
| docosahexaenoic acid + eicosapentaenoic acid | diindolylmethane | testosterone |
| docosahexaenoic acid + eicosapentaenoic acid | diindolylmethane | dehydroepiandrosterone |
| docosahexaenoic acid + eicosapentaenoic acid | diindolylmethane | androstenedione |
| docosahexaenoic acid + eicosapentaenoic acid | diindolylmethane | androstenediol |
| docosahexaenoic acid + eicosapentaenoic acid | diindolylmethane | androsterone |
| docosahexaenoic acid + eicosapentaenoic acid | diindolylmethane | dihydrotestosterone |
| gamma-linolenic acid + eicosapentaenoic acid | diindolylmethane | estrogen |
| gamma-linolenic acid + eicosapentaenoic acid | diindolylmethane | conjugated estrogens |
| gamma-linolenic acid + eicosapentaenoic acid | diindolylmethane | dienestrol |
| gamma-linolenic acid + eicosapentaenoic acid | diindolylmethane | estrone |
| gamma-linolenic acid + eicosapentaenoic acid | diindolylmethane | esterified estrogens |
| gamma-linolenic acid + eicosapentaenoic acid | diindolylmethane | estradiol |
| gamma-linolenic acid + eicosapentaenoic acid | diindolylmethane | estriol |
| gamma-linolenic acid + eicosapentaenoic acid | diindolylmethane | estropipate |
| gamma-linolenic acid + eicosapentaenoic acid | diindolylmethane | ethinyl estradiol |
| gamma-linolenic acid + eicosapentaenoic acid | diindolylmethane | testosterone |
| gamma-linolenic acid + eicosapentaenoic acid | diindolylmethane | dehydroepiandrosterone |
| gamma-linolenic acid + eicosapentaenoic acid | diindolylmethane | androstenedione |
| gamma-linolenic acid + eicosapentaenoic acid | diindolylmethane | androstenediol |
| gamma-linolenic acid + eicosapentaenoic acid | diindolylmethane | androsterone |
| gamma-linolenic acid + eicosapentaenoic acid | diindolylmethane | dihydrotestosterone |
| alpha-linolenic acid + docosahexaenoic acid + | diindolylmethane | estrogen |
| gamma-linolenic acid | | |
| alpha-linolenic acid + docosahexaenoic acid + | diindolylmethane | conjugated estrogens |
| gamma-linolenic acid | | |
| alpha-linolenic acid + docosahexaenoic acid + | diindolylmethane | dienestrol |
| gamma-linolenic acid | | |
| alpha-linolenic acid + docosahexaenoic acid + | diindolylmethane | estrone |
| gamma-linolenic acid | | |
| alpha-linolenic acid + docosahexaenoic acid + | diindolylmethane | esterified estrogens |
| gamma-linolenic acid | | |
| alpha-linolenic acid + docosahexaenoic acid + | diindolylmethane | estradiol |
| gamma-linolenic acid | | |
| alpha-linolenic acid + docosahexaenoic acid + | diindolylmethane | estriol |
| gamma-linolenic acid | | |
| alpha-linolenic acid + docosahexaenoic acid + | diindolylmethane | estropipate |
| gamma-linolenic acid | | |
| alpha-linolenic acid + docosahexaenoic acid + | diindolylmethane | ethinyl estradiol |
| gamma-linolenic acid | | |
| alpha-linolenic acid + docosahexaenoic acid + | diindolylmethane | testosterone |
| gamma-linolenic acid | | |
| alpha-linolenic acid + docosahexaenoic acid + | diindolylmethane | dehydroepiandrosterone |
| gamma-linolenic acid | | |
| alpha-linolenic acid + docosahexaenoic acid + | diindolylmethane | androstenedione |
| gamma-linolenic acid | | |
| alpha-linolenic acid + docosahexaenoic acid + | diindolylmethane | androstenediol |
| gamma-linolenic acid | | |
| alpha-linolenic acid + docosahexaenoic acid + | diindolylmethane | androsterone |
| gamma-linolenic acid | | |
| alpha-linolenic acid + docosahexaenoic acid + | diindolylmethane | dihydrotestosterone |
| gamma-linolenic acid | | |
| alpha-linolenic acid + docosahexaenoic acid + | diindolylmethane | estrogen |
| eicosapentaenoic acid | | |
| alpha-linolenic acid + docosahexaenoic acid + | diindolylmethane | conjugated estrogens |
| eicosapentaenoic acid | | |
| alpha-linolenic acid + docosahexaenoic acid + | diindolylmethane | dienestrol |
| eicosapentaenoic acid | | |
| alpha-linolenic acid + docosahexaenoic acid + | diindolylmethane | estrone |
| eicosapentaenoic acid | | |
| alpha-linolenic acid + docosahexaenoic acid + | diindolylmethane | esterified estrogens |
| eicosapentaenoic acid | | |
| alpha-linolenic acid + docosahexaenoic acid + | diindolylmethane | estradiol |
| eicosapentaenoic acid | | |
| alpha-linolenic acid + docosahexaenoic acid + | diindolylmethane | estriol |
| eicosapentaenoic acid | | |
| alpha-linolenic acid + docosahexaenoic acid + | diindolylmethane | estropipate |
| eicosapentaenoic acid | | |
| alpha-linolenic acid + docosahexaenoic acid + | diindolylmethane | ethinyl estradiol |
| eicosapentaenoic acid | | |
| alpha-linolenic acid + docosahexaenoic acid + | diindolylmethane | testosterone |
| eicosapentaenoic acid | | |
| alpha-linolenic acid + docosahexaenoic acid + | diindolylmethane | dehydroepiandrosterone |
| eicosapentaenoic acid | | |
| alpha-linolenic acid + docosahexaenoic acid + | diindolylmethane | androstenedione |
| eicosapentaenoic acid | | |
| alpha-linolenic acid + docosahexaenoic acid + | diindolylmethane | androstenediol |
| eicosapentaenoic acid | | |
| alpha-linolenic acid + docosahexaenoic acid + | diindolylmethane | androsterone |
| eicosapentaenoic acid | | |
| alpha-linolenic acid + docosahexaenoic acid + | diindolylmethane | dihydrotestosterone |
| eicosapentaenoic acid | | |
| docosahexaenoic acid + gamma-linolenic acid + | diindolylmethane | estrogen |
| eicosapentaenoic acid | | |
| docosahexaenoic acid + gamma-linolenic acid + | diindolylmethane | conjugated estrogens |
| eicosapentaenoic acid | | |
| docosahexaenoic acid + gamma-linolenic acid + | diindolylmethane | dienestrol |
| eicosapentaenoic acid | | |
| docosahexaenoic acid + gamma-linolenic acid + | diindolylmethane | estrone |
| eicosapentaenoic acid | | |
| docosahexaenoic acid + gamma-linolenic acid + | diindolylmethane | esterified estrogens |
| eicosapentaenoic acid | | |
| docosahexaenoic acid + gamma-linolenic acid + | diindolylmethane | estradiol |
| eicosapentaenoic acid | | |
| docosahexaenoic acid + gamma-linolenic acid + | diindolylmethane | estriol |
| eicosapentaenoic acid | | |
| docosahexaenoic acid + gamma-linolenic acid + | diindolylmethane | estropipate |
| eicosapentaenoic acid | | |
| docosahexaenoic acid + gamma-linolenic acid + | diindolylmethane | ethinyl estradiol |
| eicosapentaenoic acid | | |
| docosahexaenoic acid + gamma-linolenic acid + | diindolylmethane | testosterone |
| eicosapentaenoic acid | | |
| docosahexaenoic acid + gamma-linolenic acid + | diindolylmethane | dehydroepiandrosterone |
| eicosapentaenoic acid | | |
| docosahexaenoic acid + gamma-linolenic acid + | diindolylmethane | androstenedione |
| eicosapentaenoic acid | | |
| docosahexaenoic acid + gamma-linolenic acid + | diindolylmethane | androstenediol |
| eicosapentaenoic acid | | |
| docosahexaenoic acid + gamma-linolenic acid + | diindolylmethane | androsterone |
| eicosapentaenoic acid | | |
| docosahexaenoic acid + gamma-linolenic acid + | diindolylmethane | dihydrotestosterone |
| eicosapentaenoic acid | | |
| alpha-linolenic acid + gamma-linolenic acid + | diindolylmethane | estrogen |
| eicosapentaenoic acid | | |
| alpha-linolenic acid + gamma-linolenic acid + | diindolylmethane | conjugated estrogens |
| eicosapentaenoic acid | | |
| alpha-linolenic acid + gamma-linolenic acid + | diindolylmethane | dienestrol |
| eicosapentaenoic acid | | |
| alpha-linolenic acid + gamma-linolenic acid + | diindolylmethane | estrone |
| eicosapentaenoic acid | | |
| alpha-linolenic acid + gamma-linolenic acid + | diindolylmethane | esterified estrogens |
| eicosapentaenoic acid | | |
| alpha-linolenic acid + gamma-linolenic acid + | diindolylmethane | estradiol |
| eicosapentaenoic acid | | |
| alpha-linolenic acid + gamma-linolenic acid + | diindolylmethane | estriol |
| eicosapentaenoic acid | | |
| alpha-linolenic acid + gamma-linolenic acid + | diindolylmethane | estropipate |
| eicosapentaenoic acid | | |
| alpha-linolenic acid + gamma-linolenic acid + | diindolylmethane | ethinyl estradiol |
| eicosapentaenoic acid | | |
| alpha-linolenic acid + gamma-linolenic acid + | diindolylmethane | testosterone |
| eicosapentaenoic acid | | |
| alpha-linolenic acid + gamma-linolenic acid + | diindolylmethane | dehydroepiandrosterone |
| eicosapentaenoic acid | | |
| alpha-linolenic acid + gamma-linolenic acid + | diindolylmethane | androstenedione |
| eicosapentaenoic acid | | |
| alpha-linolenic acid + gamma-linolenic acid + | diindolylmethane | androstenediol |
| eicosapentaenoic acid | | |
| alpha-linolenic acid + gamma-linolenic acid + | diindolylmethane | androsterone |
| eicosapentaenoic acid | | |
| alpha-linolenic acid + gamma-linolenic acid + | diindolylmethane | dihydrotestosterone |
| eicosapentaenoic acid | | |
| alpha-linolenic acid + docosahexaenoic acid + | diindolylmethane | estrogen |
| gamma-linolenic acid + eicosapentaenoic acid | | |
| alpha-linolenic acid + docosahexaenoic acid + | diindolylmethane | conjugated estrogens |
| gamma-linolenic acid + eicosapentaenoic acid | | |
| alpha-linolenic acid + docosahexaenoic acid + | diindolylmethane | dienestrol |
| gamma-linolenic acid + eicosapentaenoic acid | | |
| alpha-linolenic acid + docosahexaenoic acid + | diindolylmethane | estrone |
| gamma-linolenic acid + eicosapentaenoic acid | | |
| alpha-linolenic acid + docosahexaenoic acid + | diindolylmethane | esterified estrogens |
| gamma-linolenic acid + eicosapentaenoic acid | | |
| alpha-linolenic acid + docosahexaenoic acid + | diindolylmethane | estradiol |
| gamma-linolenic acid + eicosapentaenoic acid | | |
| alpha-linolenic acid + docosahexaenoic acid + | diindolylmethane | estriol |
| gamma-linolenic acid + eicosapentaenoic acid | | |
| alpha-linolenic acid + docosahexaenoic acid + | diindolylmethane | estropipate |
| gamma-linolenic acid + eicosapentaenoic acid | | |
| alpha-linolenic acid + docosahexaenoic acid + | diindolylmethane | ethinyl estradiol |
| gamma-linolenic acid + eicosapentaenoic acid | | |
| alpha-linolenic acid + docosahexaenoic acid + | diindolylmethane | testosterone |
| gamma-linolenic acid + eicosapentaenoic acid | | |
| alpha-linolenic acid + docosahexaenoic acid + | diindolylmethane | dehydroepiandrosterone |
| gamma-linolenic acid + eicosapentaenoic acid | | |
| alpha-linolenic acid + docosahexaenoic acid + | diindolylmethane | androstenedione |
| gamma-linolenic acid + eicosapentaenoic acid | | |
| alpha-linolenic acid + docosahexaenoic acid + | diindolylmethane | androstenediol |
| gamma-linolenic acid + eicosapentaenoic acid | | |
| alpha-linolenic acid + docosahexaenoic acid + | diindolylmethane | androsterone |
| gamma-linolenic acid + eicosapentaenoic acid | | |
| alpha-linolenic acid + docosahexaenoic acid + | diindolylmethane | dihydrotestosterone |
| gamma-linolenic acid + eicosapentaenoic acid | | |

In an alternative exemplary embodiment, folic acid may be combined with any of the combinations delineated in Table C.

Yet a further exemplary combination comprises at least one polyunsaturated fatty acid, diindolylmethane, and a steroid. Non-limiting examples of this combination are delineated in Table D.

TABLE D

| First Agent | Second Agent | Third Agent |
|---|---|---|
| alpha-linolenic acid | diindolylmethane | dexamethasone |
| alpha-linolenic acid | diindolylmethane | hydrocortisone |
| alpha-linolenic acid | diindolylmethane | cortisone acetate |
| alpha-linolenic acid | diindolylmethane | prednisone |
| alpha-linolenic acid | diindolylmethane | prednisolone |
| alpha-linolenic acid | diindolylmethane | methylprednisolone |
| alpha-linolenic acid | diindolylmethane | betamethasone |
| alpha-linolenic acid | diindolylmethane | triamcinolone |
| alpha-linolenic acid | diindolylmethane | beclometasone |
| alpha-linolenic acid | diindolylmethane | fludrocortisone acetate |
| docosahexaenoic acid | diindolylmethane | dexamethasone |
| docosahexaenoic acid | diindolylmethane | hydrocortisone |
| docosahexaenoic acid | diindolylmethane | cortisone acetate |
| docosahexaenoic acid | diindolylmethane | prednisone |
| docosahexaenoic acid | diindolylmethane | prednisolone |
| docosahexaenoic acid | diindolylmethane | methylprednisolone |
| docosahexaenoic acid | diindolylmethane | betamethasone |
| docosahexaenoic acid | diindolylmethane | triamcinolone |
| docosahexaenoic acid | diindolylmethane | beclometasone |
| docosahexaenoic acid | diindolylmethane | fludrocortisone acetate |
| gamma-linolenic acid | diindolylmethane | dexamethasone |
| gamma-linolenic acid | diindolylmethane | hydrocortisone |
| gamma-linolenic acid | diindolylmethane | cortisone acetate |
| gamma-linolenic acid | diindolylmethane | prednisone |
| gamma-linolenic acid | diindolylmethane | prednisolone |
| gamma-linolenic acid | diindolylmethane | methylprednisolone |
| gamma-linolenic acid | diindolylmethane | betamethasone |
| gamma-linolenic acid | diindolylmethane | triamcinolone |
| gamma-linolenic acid | diindolylmethane | beclometasone |
| gamma-linolenic acid | diindolylmethane | fludrocortisone acetate |
| eicosapentaenoic acid | diindolylmethane | dexamethasone |
| eicosapentaenoic acid | diindolylmethane | hydrocortisone |
| eicosapentaenoic acid | diindolylmethane | cortisone acetate |
| eicosapentaenoic acid | diindolylmethane | prednisone |
| eicosapentaenoic acid | diindolylmethane | prednisolone |
| eicosapentaenoic acid | diindolylmethane | methylprednisolone |
| eicosapentaenoic acid | diindolylmethane | betamethasone |
| eicosapentaenoic acid | diindolylmethane | triamcinolone |
| eicosapentaenoic acid | diindolylmethane | beclometasone |
| eicosapentaenoic acid | diindolylmethane | fludrocortisone acetate |
| alpha-linolenic acid + docosahexaenoic acid | diindolylmethane | dexamethasone |
| alpha-linolenic acid + docosahexaenoic acid | diindolylmethane | hydrocortisone |
| alpha-linolenic acid + docosahexaenoic acid | diindolylmethane | cortisone acetate |
| alpha-linolenic acid + docosahexaenoic acid | diindolylmethane | prednisone |
| alpha-linolenic acid + docosahexaenoic acid | diindolylmethane | prednisolone |
| alpha-linolenic acid + docosahexaenoic acid | diindolylmethane | methylprednisolone |
| alpha-linolenic acid + docosahexaenoic acid | diindolylmethane | betamethasone |
| alpha-linolenic acid + docosahexaenoic acid | diindolylmethane | triamcinolone |
| alpha-linolenic acid + docosahexaenoic acid | diindolylmethane | beclometasone |
| alpha-linolenic acid + docosahexaenoic acid | diindolylmethane | fludrocortisone acetate |
| alpha-linolenic acid + gamma-linolenic acid | diindolylmethane | dexamethasone |
| alpha-linolenic acid + gamma-linolenic acid | diindolylmethane | hydrocortisone |
| alpha-linolenic acid + gamma-linolenic acid | diindolylmethane | cortisone acetate |
| alpha-linolenic acid + gamma-linolenic acid | diindolylmethane | prednisone |
| alpha-linolenic acid + gamma-linolenic acid | diindolylmethane | prednisolone |
| alpha-linolenic acid + gamma-linolenic acid | diindolylmethane | methylprednisolone |
| alpha-linolenic acid + gamma-linolenic acid | diindolylmethane | betamethasone |
| alpha-linolenic acid + gamma-linolenic acid | diindolylmethane | triamcinolone |
| alpha-linolenic acid + gamma-linolenic acid | diindolylmethane | beclometasone |
| alpha-linolenic acid + gamma-linolenic acid | diindolylmethane | fludrocortisone acetate |
| alpha-linolenic acid + eicosapentaenoic acid | diindolylmethane | dexamethasone |
| alpha-linolenic acid + eicosapentaenoic acid | diindolylmethane | hydrocortisone |
| alpha-linolenic acid + eicosapentaenoic acid | diindolylmethane | cortisone acetate |
| alpha-linolenic acid + eicosapentaenoic acid | diindolylmethane | prednisone |
| alpha-linolenic acid + eicosapentaenoic acid | diindolylmethane | prednisolone |
| alpha-linolenic acid + eicosapentaenoic acid | diindolylmethane | methylprednisolone |
| alpha-linolenic acid + eicosapentaenoic acid | diindolylmethane | betamethasone |
| alpha-linolenic acid + eicosapentaenoic acid | diindolylmethane | triamcinolone |
| alpha-linolenic acid + eicosapentaenoic acid | diindolylmethane | beclometasone |
| alpha-linolenic acid + eicosapentaenoic acid | diindolylmethane | fludrocortisone acetate |
| docosahexaenoic acid + gamma-linolenic acid | diindolylmethane | dexamethasone |
| docosahexaenoic acid + gamma-linolenic acid | diindolylmethane | hydrocortisone |
| docosahexaenoic acid + gamma-linolenic acid | diindolylmethane | cortisone acetate |
| docosahexaenoic acid + gamma-linolenic acid | diindolylmethane | prednisone |
| docosahexaenoic acid + gamma-linolenic acid | diindolylmethane | prednisolone |
| docosahexaenoic acid + gamma-linolenic acid | diindolylmethane | methylprednisolone |
| docosahexaenoic acid + gamma-linolenic acid | diindolylmethane | betamethasone |
| docosahexaenoic acid + gamma-linolenic acid | diindolylmethane | triamcinolone |
| docosahexaenoic acid + gamma-linolenic acid | diindolylmethane | beclometasone |
| docosahexaenoic acid + gamma-linolenic acid | diindolylmethane | fludrocortisone acetate |
| docosahexaenoic acid + eicosapentaenoic acid | diindolylmethane | dexamethasone |
| docosahexaenoic acid + eicosapentaenoic acid | diindolylmethane | hydrocortisone |
| docosahexaenoic acid + eicosapentaenoic acid | diindolylmethane | cortisone acetate |
| docosahexaenoic acid + eicosapentaenoic acid | diindolylmethane | prednisone |
| docosahexaenoic acid + eicosapentaenoic acid | diindolylmethane | prednisolone |
| docosahexaenoic acid + eicosapentaenoic acid | diindolylmethane | methylprednisolone |
| docosahexaenoic acid + eicosapentaenoic acid | diindolylmethane | betamethasone |
| docosahexaenoic acid + eicosapentaenoic acid | diindolylmethane | triamcinolone |
| docosahexaenoic acid + eicosapentaenoic acid | diindolylmethane | beclometasone |
| docosahexaenoic acid + eicosapentaenoic acid | diindolylmethane | fludrocortisone acetate |
| gamma-linolenic acid + eicosapentaenoic acid | diindolylmethane | dexamethasone |
| gamma-linolenic acid + eicosapentaenoic acid | diindolylmethane | hydrocortisone |
| gamma-linolenic acid + eicosapentaenoic acid | diindolylmethane | cortisone acetate |
| gamma-linolenic acid + eicosapentaenoic acid | diindolylmethane | prednisone |
| gamma-linolenic acid + eicosapentaenoic acid | diindolylmethane | prednisolone |
| gamma-linolenic acid + eicosapentaenoic acid | diindolylmethane | methylprednisolone |
| gamma-linolenic acid + eicosapentaenoic acid | diindolylmethane | betamethasone |
| gamma-linolenic acid + eicosapentaenoic acid | diindolylmethane | triamcinolone |
| gamma-linolenic acid + eicosapentaenoic acid | diindolylmethane | beclometasone |
| gamma-linolenic acid + eicosapentaenoic acid | diindolylmethane | fludrocortisone acetate |
| alpha-linolenic acid + docosahexaenoic acid + | diindolylmethane | dexamethasone |
| gamma-linolenic acid | | |
| alpha-linolenic acid + docosahexaenoic acid + | diindolylmethane | hydrocortisone |
| gamma-linolenic acid | | |
| alpha-linolenic acid + docosahexaenoic acid + | diindolylmethane | cortisone acetate |
| gamma-linolenic acid | | |
| alpha-linolenic acid + docosahexaenoic acid + | diindolylmethane | prednisone |
| gamma-linolenic acid | | |
| alpha-linolenic acid + docosahexaenoic acid + | diindolylmethane | prednisolone |
| gamma-linolenic acid | | |
| alpha-linolenic acid + docosahexaenoic acid + | diindolylmethane | methylprednisolone |
| gamma-linolenic acid | | |
| alpha-linolenic acid + docosahexaenoic acid + | diindolylmethane | betamethasone |
| gamma-linolenic acid | | |
| alpha-linolenic acid + docosahexaenoic acid + | diindolylmethane | triamcinolone |
| gamma-linolenic acid | | |
| alpha-linolenic acid + docosahexaenoic acid + | diindolylmethane | beclometasone |
| gamma-linolenic acid | | |
| alpha-linolenic acid + docosahexaenoic acid + | diindolylmethane | fludrocortisone acetate |
| gamma-linolenic acid | | |
| alpha-linolenic acid + docosahexaenoic acid + | diindolylmethane | dexamethasone |
| eicosapentaenoic acid | | |
| alpha-linolenic acid + docosahexaenoic acid + | diindolylmethane | hydrocortisone |
| eicosapentaenoic acid | | |
| alpha-linolenic acid + docosahexaenoic acid + | diindolylmethane | cortisone acetate |
| eicosapentaenoic acid | | |
| alpha-linolenic acid + docosahexaenoic acid + | diindolylmethane | prednisone |
| eicosapentaenoic acid | | |
| alpha-linolenic acid + docosahexaenoic acid + | diindolylmethane | prednisolone |
| eicosapentaenoic acid | | |
| alpha-linolenic acid + docosahexaenoic acid + | diindolylmethane | methylprednisolone |
| eicosapentaenoic acid | | |
| alpha-linolenic acid + docosahexaenoic acid + | diindolylmethane | betamethasone |
| eicosapentaenoic acid | | |
| alpha-linolenic acid + docosahexaenoic acid + | diindolylmethane | triamcinolone |
| eicosapentaenoic acid | | |
| alpha-linolenic acid + docosahexaenoic acid + | diindolylmethane | beclometasone |
| eicosapentaenoic acid | | |
| alpha-linolenic acid + docosahexaenoic acid + | diindolylmethane | fludrocortisone acetate |
| eicosapentaenoic acid | | |
| docosahexaenoic acid + gamma-linolenic acid + | diindolylmethane | dexamethasone |
| eicosapentaenoic acid | | |
| docosahexaenoic acid + gamma-linolenic acid + | diindolylmethane | hydrocortisone |
| eicosapentaenoic acid | | |
| docosahexaenoic acid + gamma-linolenic acid + | diindolylmethane | cortisone acetate |
| eicosapentaenoic acid | | |
| docosahexaenoic acid + gamma-linolenic acid + | diindolylmethane | prednisone |
| eicosapentaenoic acid | | |
| docosahexaenoic acid + gamma-linolenic acid + | diindolylmethane | prednisolone |
| eicosapentaenoic acid | | |
| docosahexaenoic acid + gamma-linolenic acid + | diindolylmethane | methylprednisolone |
| eicosapentaenoic acid | | |
| docosahexaenoic acid + gamma-linolenic acid + | diindolylmethane | betamethasone |
| eicosapentaenoic acid | | |
| docosahexaenoic acid + gamma-linolenic acid + | diindolylmethane | triamcinolone |
| eicosapentaenoic acid | | |
| docosahexaenoic acid + gamma-linolenic acid + | diindolylmethane | beclometasone |
| eicosapentaenoic acid | | |
| docosahexaenoic acid + gamma-linolenic acid + | diindolylmethane | fludrocortisone acetate |
| eicosapentaenoic acid | | |
| alpha-linolenic acid + gamma-linolenic acid + | diindolylmethane | dexamethasone |
| eicosapentaenoic acid | | |
| alpha-linolenic acid + gamma-linolenic acid + | diindolylmethane | hydrocortisone |
| eicosapentaenoic acid | | |
| alpha-linolenic acid + gamma-linolenic acid + | diindolylmethane | cortisone acetate |
| eicosapentaenoic acid | | |
| alpha-linolenic acid + gamma-linolenic acid + | diindolylmethane | prednisone |
| eicosapentaenoic acid | | |
| alpha-linolenic acid + gamma-linolenic acid + | diindolylmethane | prednisolone |
| eicosapentaenoic acid | | |
| alpha-linolenic acid + gamma-linolenic acid + | diindolylmethane | methylprednisolone |
| eicosapentaenoic acid | | |
| alpha-linolenic acid + gamma-linolenic acid + | diindolylmethane | betamethasone |
| eicosapentaenoic acid | | |
| alpha-linolenic acid + gamma-linolenic acid + | diindolylmethane | triamcinolone |
| eicosapentaenoic acid | | |
| alpha-linolenic acid + gamma-linolenic acid + | diindolylmethane | beclometasone |
| eicosapentaenoic acid | | |
| alpha-linolenic acid + gamma-linolenic acid + | diindolylmethane | fludrocortisone acetate |
| eicosapentaenoic acid | | |
| alpha-linolenic acid + docosahexaenoic acid + | diindolylmethane | dexamethasone |
| gamma-linolenic acid + eicosapentaenoic acid | | |
| alpha-linolenic acid + docosahexaenoic acid + | diindolylmethane | hydrocortisone |
| gamma-linolenic acid + eicosapentaenoic acid | | |
| alpha-linolenic acid + docosahexaenoic acid + | diindolylmethane | cortisone acetate |
| gamma-linolenic acid + eicosapentaenoic acid | | |
| alpha-linolenic acid + docosahexaenoic acid + | diindolylmethane | prednisone |
| gamma-linolenic acid + eicosapentaenoic acid | | |
| alpha-linolenic acid + docosahexaenoic acid + | diindolylmethane | prednisolone |
| gamma-linolenic acid + eicosapentaenoic acid | | |
| alpha-linolenic acid + docosahexaenoic acid + | diindolylmethane | methylprednisolone |
| gamma-linolenic acid + eicosapentaenoic acid | | |
| alpha-linolenic acid + docosahexaenoic acid + | diindolylmethane | betamethasone |
| gamma-linolenic acid + eicosapentaenoic acid | | |
| alpha-linolenic acid + docosahexaenoic acid + | diindolylmethane | triamcinolone |
| gamma-linolenic acid + eicosapentaenoic acid | | |
| alpha-linolenic acid + docosahexaenoic acid + | diindolylmethane | beclometasone |
| gamma-linolenic acid + eicosapentaenoic acid | | |
| alpha-linolenic acid + docosahexaenoic acid + | diindolylmethane | fludrocortisone acetate |
| gamma-linolenic acid + eicosapentaenoic acid | | |

In an alternative exemplary embodiment, folic acid may be combined with any of the combinations delineated in Table D.

An additional exemplary combination comprises at least one polyunsaturated fatty acid, diindolylmethane, and a HMG-CoA reductase inhibitor. Non-limiting examples of this combination are delineated in Table E.

TABLE E

| First Agent | Second Agent | Third Agent |
|---|---|---|
| alpha-linolenic acid | diindolylmethane | mevastatin |
| alpha-linolenic acid | diindolylmethane | atorvastatin |
| alpha-linolenic acid | diindolylmethane | cerivastatin |
| alpha-linolenic acid | diindolylmethane | fluvastatin |
| alpha-linolenic acid | diindolylmethane | pitavastatin |
| alpha-linolenic acid | diindolylmethane | rosuvastatin |
| alpha-linolenic acid | diindolylmethane | simvastatin |
| alpha-linolenic acid | diindolylmethane | lovastatin |
| alpha-linolenic acid | diindolylmethane | pravastatin |
| docosahexaenoic acid | diindolylmethane | mevastatin |
| docosahexaenoic acid | diindolylmethane | atorvastatin |
| docosahexaenoic acid | diindolylmethane | cerivastatin |
| docosahexaenoic acid | diindolylmethane | fluvastatin |
| docosahexaenoic acid | diindolylmethane | pitavastatin |
| docosahexaenoic acid | diindolylmethane | rosuvastatin |
| docosahexaenoic acid | diindolylmethane | simvastatin |
| docosahexaenoic acid | diindolylmethane | lovastatin |
| docosahexaenoic acid | diindolylmethane | pravastatin |
| gamma-linolenic acid | diindolylmethane | mevastatin |
| gamma-linolenic acid | diindolylmethane | atorvastatin |
| gamma-linolenic acid | diindolylmethane | cerivastatin |
| gamma-linolenic acid | diindolylmethane | fluvastatin |
| gamma-linolenic acid | diindolylmethane | pitavastatin |
| gamma-linolenic acid | diindolylmethane | rosuvastatin |
| gamma-linolenic acid | diindolylmethane | simvastatin |
| gamma-linolenic acid | diindolylmethane | lovastatin |
| gamma-linolenic acid | diindolylmethane | pravastatin |
| eicosapentaenoic acid | diindolylmethane | mevastatin |
| eicosapentaenoic acid | diindolylmethane | atorvastatin |
| eicosapentaenoic acid | diindolylmethane | cerivastatin |
| eicosapentaenoic acid | diindolylmethane | fluvastatin |
| eicosapentaenoic acid | diindolylmethane | pitavastatin |
| eicosapentaenoic acid | diindolylmethane | rosuvastatin |
| eicosapentaenoic acid | diindolylmethane | simvastatin |
| eicosapentaenoic acid | diindolylmethane | lovastatin |
| eicosapentaenoic acid | diindolylmethane | pravastatin |
| alpha-linolenic acid + docosahexaenoic acid | diindolylmethane | mevastatin |
| alpha-linolenic acid + docosahexaenoic acid | diindolylmethane | atorvastatin |
| alpha-linolenic acid + docosahexaenoic acid | diindolylmethane | cerivastatin |
| alpha-linolenic acid + docosahexaenoic acid | diindolylmethane | fluvastatin |
| alpha-linolenic acid + docosahexaenoic acid | diindolylmethane | pitavastatin |
| alpha-linolenic acid + docosahexaenoic acid | diindolylmethane | rosuvastatin |
| alpha-linolenic acid + docosahexaenoic acid | diindolylmethane | simvastatin |
| alpha-linolenic acid + docosahexaenoic acid | diindolylmethane | lovastatin |
| alpha-linolenic acid + docosahexaenoic acid | diindolylmethane | pravastatin |
| alpha-linolenic acid + gamma-linolenic acid | diindolylmethane | mevastatin |
| alpha-linolenic acid + gamma-linolenic acid | diindolylmethane | atorvastatin |
| alpha-linolenic acid + gamma-linolenic acid | diindolylmethane | cerivastatin |
| alpha-linolenic acid + gamma-linolenic acid | diindolylmethane | fluvastatin |
| alpha-linolenic acid + gamma-linolenic acid | diindolylmethane | pitavastatin |
| alpha-linolenic acid + gamma-linolenic acid | diindolylmethane | rosuvastatin |
| alpha-linolenic acid + gamma-linolenic acid | diindolylmethane | simvastatin |
| alpha-linolenic acid + gamma-linolenic acid | diindolylmethane | lovastatin |
| alpha-linolenic acid + gamma-linolenic acid | diindolylmethane | pravastatin |
| alpha-linolenic acid + eicosapentaenoic acid | diindolylmethane | mevastatin |
| alpha-linolenic acid + eicosapentaenoic acid | diindolylmethane | atorvastatin |
| alpha-linolenic acid + eicosapentaenoic acid | diindolylmethane | cerivastatin |
| alpha-linolenic acid + eicosapentaenoic acid | diindolylmethane | fluvastatin |
| alpha-linolenic acid + eicosapentaenoic acid | diindolylmethane | pitavastatin |
| alpha-linolenic acid + eicosapentaenoic acid | diindolylmethane | rosuvastatin |
| alpha-linolenic acid + eicosapentaenoic acid | diindolylmethane | simvastatin |
| alpha-linolenic acid + eicosapentaenoic acid | diindolylmethane | lovastatin |
| alpha-linolenic acid + eicosapentaenoic acid | diindolylmethane | pravastatin |
| docosahexaenoic acid + gamma-linolenic acid | diindolylmethane | mevastatin |
| docosahexaenoic acid + gamma-linolenic acid | diindolylmethane | atorvastatin |
| docosahexaenoic acid + gamma-linolenic acid | diindolylmethane | cerivastatin |
| docosahexaenoic acid + gamma-linolenic acid | diindolylmethane | fluvastatin |
| docosahexaenoic acid + gamma-linolenic acid | diindolylmethane | pitavastatin |
| docosahexaenoic acid + gamma-linolenic acid | diindolylmethane | rosuvastatin |
| docosahexaenoic acid + gamma-linolenic acid | diindolylmethane | simvastatin |
| docosahexaenoic acid + gamma-linolenic acid | diindolylmethane | lovastatin |
| docosahexaenoic acid + gamma-linolenic acid | diindolylmethane | pravastatin |
| docosahexaenoic acid + eicosapentaenoic acid | diindolylmethane | mevastatin |
| docosahexaenoic acid + eicosapentaenoic acid | diindolylmethane | atorvastatin |
| docosahexaenoic acid + eicosapentaenoic acid | diindolylmethane | cerivastatin |
| docosahexaenoic acid + eicosapentaenoic acid | diindolylmethane | fluvastatin |
| docosahexaenoic acid + eicosapentaenoic acid | diindolylmethane | pitavastatin |
| docosahexaenoic acid + eicosapentaenoic acid | diindolylmethane | rosuvastatin |
| docosahexaenoic acid + eicosapentaenoic acid | diindolylmethane | simvastatin |
| docosahexaenoic acid + eicosapentaenoic acid | diindolylmethane | lovastatin |
| docosahexaenoic acid + eicosapentaenoic acid | diindolylmethane | pravastatin |
| gamma-linolenic acid + eicosapentaenoic acid | diindolylmethane | mevastatin |
| gamma-linolenic acid + eicosapentaenoic acid | diindolylmethane | atorvastatin |
| gamma-linolenic acid + eicosapentaenoic acid | diindolylmethane | cerivastatin |
| gamma-linolenic acid + eicosapentaenoic acid | diindolylmethane | fluvastatin |
| gamma-linolenic acid + eicosapentaenoic acid | diindolylmethane | pitavastatin |
| gamma-linolenic acid + eicosapentaenoic acid | diindolylmethane | rosuvastatin |
| gamma-linolenic acid + eicosapentaenoic acid | diindolylmethane | simvastatin |
| gamma-linolenic acid + eicosapentaenoic acid | diindolylmethane | lovastatin |
| gamma-linolenic acid + eicosapentaenoic acid | diindolylmethane | pravastatin |
| alpha-linolenic acid + docosahexaenoic acid + | diindolylmethane | mevastatin |
| gamma-linolenic acid | | |
| alpha-linolenic acid + docosahexaenoic acid + | diindolylmethane | atorvastatin |
| gamma-linolenic acid | | |
| alpha-linolenic acid + docosahexaenoic acid + | diindolylmethane | cerivastatin |
| gamma-linolenic acid | | |
| alpha-linolenic acid + docosahexaenoic acid + | diindolylmethane | fluvastatin |
| gamma-linolenic acid | | |
| alpha-linolenic acid + docosahexaenoic acid + | diindolylmethane | pitavastatin |
| gamma-linolenic acid | | |
| alpha-linolenic acid + docosahexaenoic acid + | diindolylmethane | rosuvastatin |
| gamma-linolenic acid | | |
| alpha-linolenic acid + docosahexaenoic acid + | diindolylmethane | simvastatin |
| gamma-linolenic acid | | |
| alpha-linolenic acid + docosahexaenoic acid + | diindolylmethane | lovastatin |
| gamma-linolenic acid | | |
| alpha-linolenic acid + docosahexaenoic acid + | diindolylmethane | pravastatin |
| gamma-linolenic acid | | |
| alpha-linolenic acid + docosahexaenoic acid + eicosapentaenoic | diindolylmethane | mevastatin |
| acid | | |
| alpha-linolenic acid + docosahexaenoic acid + | diindolylmethane | atorvastatin |
| eicosapentaenoic acid | | |
| alpha-linolenic acid + docosahexaenoic acid + eicosapentaenoic | diindolylmethane | cerivastatin |
| acid | | |
| alpha-linolenic acid + docosahexaenoic acid + | diindolylmethane | fluvastatin |
| eicosapentaenoic acid | | |
| alpha-linolenic acid + docosahexaenoic acid + | diindolylmethane | pitavastatin |
| eicosapentaenoic acid | | |
| alpha-linolenic acid + docosahexaenoic acid + | diindolylmethane | rosuvastatin |
| eicosapentaenoic acid | | |
| alpha-linolenic acid + docosahexaenoic acid + eicosapentaenoic | diindolylmethane | simvastatin |
| acid | | |
| alpha-linolenic acid + docosahexaenoic acid + | diindolylmethane | lovastatin |
| eicosapentaenoic acid | | |
| alpha-linolenic acid + docosahexaenoic acid + | diindolylmethane | pravastatin |
| eicosapentaenoic acid | | |
| docosahexaenoic acid + gamma-linolenic acid + | diindolylmethane | mevastatin |
| eicosapentaenoic acid | | |
| docosahexaenoic acid + gamma-linolenic acid + | diindolylmethane | atorvastatin |
| eicosapentaenoic acid | | |
| docosahexaenoic acid + gamma-linolenic acid + | diindolylmethane | cerivastatin |
| eicosapentaenoic acid | | |
| docosahexaenoic acid + gamma-linolenic acid + | diindolylmethane | fluvastatin |
| eicosapentaenoic acid | | |
| docosahexaenoic acid + gamma-linolenic acid + | diindolylmethane | pitavastatin |
| eicosapentaenoic acid | | |
| docosahexaenoic acid + gamma-linolenic acid + | diindolylmethane | rosuvastatin |
| eicosapentaenoic acid | | |
| docosahexaenoic acid + gamma-linolenic acid + | diindolylmethane | simvastatin |
| eicosapentaenoic acid | | |
| docosahexaenoic acid + gamma-linolenic acid + | diindolylmethane | lovastatin |
| eicosapentaenoic acid | | |
| docosahexaenoic acid + gamma-linolenic acid + | diindolylmethane | pravastatin |
| eicosapentaenoic acid | | |
| alpha-linolenic acid + gamma-linolenic acid + | diindolylmethane | mevastatin |
| eicosapentaenoic acid | | |
| alpha-linolenic acid + gamma-linolenic acid + | diindolylmethane | atorvastatin |
| eicosapentaenoic acid | | |
| alpha-linolenic acid + gamma-linolenic acid + | diindolylmethane | cerivastatin |
| eicosapentaenoic acid | | |
| alpha-linolenic acid + gamma-linolenic acid + | diindolylmethane | fluvastatin |
| eicosapentaenoic acid | | |
| alpha-linolenic acid + gamma-linolenic acid + | diindolylmethane | pitavastatin |
| eicosapentaenoic acid | | |
| alpha-linolenic acid + gamma-linolenic acid + | diindolylmethane | rosuvastatin |
| eicosapentaenoic acid | | |
| alpha-linolenic acid + gamma-linolenic acid + | diindolylmethane | simvastatin |
| eicosapentaenoic acid | | |
| alpha-linolenic acid + gamma-linolenic acid + | diindolylmethane | lovastatin |
| eicosapentaenoic acid | | |
| alpha-linolenic acid + gamma-linolenic acid + | diindolylmethane | pravastatin |
| eicosapentaenoic acid | | |
| alpha-linolenic acid + docosahexaenoic acid + | diindolylmethane | mevastatin |
| gamma-linolenic acid + eicosapentaenoic acid | | |
| alpha-linolenic acid + docosahexaenoic acid + | diindolylmethane | atorvastatin |
| gamma-linolenic acid + eicosapentaenoic acid | | |
| alpha-linolenic acid + docosahexaenoic acid + | diindolylmethane | cerivastatin |
| gamma-linolenic acid + eicosapentaenoic acid | | |
| alpha-linolenic acid + docosahexaenoic acid + | diindolylmethane | fluvastatin |
| gamma-linolenic acid + eicosapentaenoic acid | | |
| alpha-linolenic acid + docosahexaenoic acid + | diindolylmethane | pitavastatin |
| gamma-linolenic acid + eicosapentaenoic acid | | |
| alpha-linolenic acid + docosahexaenoic acid + | diindolylmethane | rosuvastatin |
| gamma-linolenic acid + eicosapentaenoic acid | | |
| alpha-linolenic acid + docosahexaenoic acid + | diindolylmethane | simvastatin |
| gamma-linolenic acid + eicosapentaenoic acid | | |
| alpha-linolenic acid + docosahexaenoic acid + | diindolylmethane | lovastatin |
| gamma-linolenic acid + eicosapentaenoic acid | | |
| alpha-linolenic acid + docosahexaenoic acid + | diindolylmethane | pravastatin |
| gamma-linolenic acid + eicosapentaenoic acid | | |

In an alternative exemplary embodiment, folic acid may be combined with any of the combinations delineated in Table E.

Yet a further exemplary combination comprises at least one polyunsaturated fatty acid, diindolylmethane, and TZD. Non-limiting examples of this combination are delineated in Table F.

TABLE F

| First Agent | Second Agent | Third Agent |
|---|---|---|
| alpha-linolenic acid | diindolylmethane | exenatide |
| alpha-linolenic acid | diindolylmethane | troglitazone |
| alpha-linolenic acid | diindolylmethane | rosiglitazone |
| alpha-linolenic acid | diindolylmethane | pioglitazone |
| alpha-linolenic acid | diindolylmethane | muraglitizar |
| alpha-linolenic acid | diindolylmethane | naveglitizar |
| docosahexaenoic acid | diindolylmethane | exenatide |
| docosahexaenoic acid | diindolylmethane | troglitazone |
| docosahexaenoic acid | diindolylmethane | rosiglitazone |
| docosahexaenoic acid | diindolylmethane | pioglitazone |
| docosahexaenoic acid | diindolylmethane | muraglitizar |
| docosahexaenoic acid | diindolylmethane | naveglitizar |
| gamma-linolenic acid | diindolylmethane | exenatide |
| gamma-linolenic acid | diindolylmethane | troglitazone |
| gamma-linolenic acid | diindolylmethane | rosiglitazone |
| gamma-linolenic acid | diindolylmethane | pioglitazone |
| gamma-linolenic acid | diindolylmethane | muraglitizar |
| gamma-linolenic acid | diindolylmethane | naveglitizar |
| eicosapentaenoic acid | diindolylmethane | exenatide |
| eicosapentaenoic acid | diindolylmethane | troglitazone |
| eicosapentaenoic acid | diindolylmethane | rosiglitazone |
| eicosapentaenoic acid | diindolylmethane | pioglitazone |
| eicosapentaenoic acid | diindolylmethane | muraglitizar |
| eicosapentaenoic acid | diindolylmethane | naveglitizar |
| alpha-linolenic acid + docosahexaenoic acid | diindolylmethane | exenatide |
| alpha-linolenic acid + docosahexaenoic acid | diindolylmethane | troglitazone |
| alpha-linolenic acid + docosahexaenoic acid | diindolylmethane | rosiglitazone |
| alpha-linolenic acid + docosahexaenoic acid | diindolylmethane | pioglitazone |
| alpha-linolenic acid + docosahexaenoic acid | diindolylmethane | muraglitizar |
| alpha-linolenic acid + docosahexaenoic acid | diindolylmethane | naveglitizar |
| alpha-linolenic acid + gamma-linolenic acid | diindolylmethane | exenatide |
| alpha-linolenic acid + gamma-linolenic acid | diindolylmethane | troglitazone |
| alpha-linolenic acid + gamma-linolenic acid | diindolylmethane | rosiglitazone |
| alpha-linolenic acid + gamma-linolenic acid | diindolylmethane | pioglitazone |
| alpha-linolenic acid + gamma-linolenic acid | diindolylmethane | muraglitizar |
| alpha-linolenic acid + gamma-linolenic acid | diindolylmethane | naveglitizar |
| alpha-linolenic acid + eicosapentaenoic acid | diindolylmethane | exenatide |
| alpha-linolenic acid + eicosapentaenoic acid | diindolylmethane | troglitazone |
| alpha-linolenic acid + eicosapentaenoic acid | diindolylmethane | rosiglitazone |
| alpha-linolenic acid + eicosapentaenoic acid | diindolylmethane | pioglitazone |
| alpha-linolenic acid + eicosapentaenoic acid | diindolylmethane | muraglitizar |
| alpha-linolenic acid + eicosapentaenoic acid | diindolylmethane | naveglitizar |
| docosahexaenoic acid + gamma-linolenic acid | diindolylmethane | exenatide |
| docosahexaenoic acid + gamma-linolenic acid | diindolylmethane | troglitazone |
| docosahexaenoic acid + gamma-linolenic acid | diindolylmethane | rosiglitazone |
| docosahexaenoic acid + gamma-linolenic acid | diindolylmethane | pioglitazone |
| docosahexaenoic acid + gamma-linolenic acid | diindolylmethane | muraglitizar |
| docosahexaenoic acid + gamma-linolenic acid | diindolylmethane | naveglitizar |
| docosahexaenoic acid + eicosapentaenoic acid | diindolylmethane | exenatide |
| docosahexaenoic acid + eicosapentaenoic acid | diindolylmethane | troglitazone |
| docosahexaenoic acid + eicosapentaenoic acid | diindolylmethane | rosiglitazone |
| docosahexaenoic acid + eicosapentaenoic acid | diindolylmethane | pioglitazone |
| docosahexaenoic acid + eicosapentaenoic acid | diindolylmethane | muraglitizar |
| docosahexaenoic acid + eicosapentaenoic acid | diindolylmethane | naveglitizar |
| gamma-linolenic acid + eicosapentaenoic acid | diindolylmethane | exenatide |
| gamma-linolenic acid + eicosapentaenoic acid | diindolylmethane | troglitazone |
| gamma-linolenic acid + eicosapentaenoic acid | diindolylmethane | rosiglitazone |
| gamma-linolenic acid + eicosapentaenoic acid | diindolylmethane | pioglitazone |
| gamma-linolenic acid + eicosapentaenoic acid | diindolylmethane | muraglitizar |
| gamma-linolenic acid + eicosapentaenoic acid | diindolylmethane | naveglitizar |
| alpha-linolenic acid + docosahexaenoic acid + | diindolylmethane | exenatide |
| gamma-linolenic acid | | |
| alpha-linolenic acid + docosahexaenoic acid + | diindolylmethane | troglitazone |
| gamma-linolenic acid | | |
| alpha-linolenic acid + docosahexaenoic acid + | diindolylmethane | rosiglitazone |
| gamma-linolenic acid | | |
| alpha-linolenic acid + docosahexaenoic acid + | diindolylmethane | pioglitazone |
| gamma-linolenic acid | | |
| alpha-linolenic acid + docosahexaenoic acid + | diindolylmethane | muraglitizar |
| gamma-linolenic acid | | |
| alpha-linolenic acid + docosahexaenoic acid + | diindolylmethane | naveglitizar |
| gamma-linolenic acid | | |
| alpha-linolenic acid + docosahexaenoic acid + | diindolylmethane | exenatide |
| eicosapentaenoic acid | | |
| alpha-linolenic acid + docosahexaenoic acid + | diindolylmethane | troglitazone |
| eicosapentaenoic acid | | |
| alpha-linolenic acid + docosahexaenoic acid + | diindolylmethane | rosiglitazone |
| eicosapentaenoic acid | | |
| alpha-linolenic acid + docosahexaenoic acid + | diindolylmethane | pioglitazone |
| eicosapentaenoic acid | | |
| alpha-linolenic acid + docosahexaenoic acid + | diindolylmethane | muraglitizar |
| eicosapentaenoic acid | | |
| alpha-linolenic acid + docosahexaenoic acid + | diindolylmethane | naveglitizar |
| eicosapentaenoic acid | | |
| docosahexaenoic acid + gamma-linolenic acid + eicosapentaenoic | diindolylmethane | exenatide |
| acid | | |
| docosahexaenoic acid + gamma-linolenic acid + | diindolylmethane | troglitazone |
| eicosapentaenoic acid | | |
| docosahexaenoic acid + gamma-linolenic acid + eicosapentaenoic | diindolylmethane | rosiglitazone |
| acid | | |
| docosahexaenoic acid + gamma-linolenic acid + | diindolylmethane | pioglitazone |
| eicosapentaenoic acid | | |
| docosahexaenoic acid + gamma-linolenic acid + | diindolylmethane | muraglitizar |
| eicosapentaenoic acid | | |
| docosahexaenoic acid + gamma-linolenic acid + | diindolylmethane | naveglitizar |
| eicosapentaenoic acid | | |
| alpha-linolenic acid + gamma-linolenic acid + | diindolylmethane | exenatide |
| eicosapentaenoic acid | | |
| alpha-linolenic acid + gamma-linolenic acid + | diindolylmethane | troglitazone |
| eicosapentaenoic acid | | |
| alpha-linolenic acid + gamma-linolenic acid + | diindolylmethane | rosiglitazone |
| eicosapentaenoic acid | | |
| alpha-linolenic acid + gamma-linolenic acid + | diindolylmethane | pioglitazone |
| eicosapentaenoic acid | | |
| alpha-linolenic acid + gamma-linolenic acid + | diindolylmethane | muraglitizar |
| eicosapentaenoic acid | | |
| alpha-linolenic acid + gamma-linolenic acid + eicosapentaenoic | diindolylmethane | naveglitizar |
| acid | | |
| alpha-linolenic acid + docosahexaenoic acid + | diindolylmethane | exenatide |
| gamma-linolenic acid + eicosapentaenoic acid | | |
| alpha-linolenic acid + docosahexaenoic acid + | | troglitazone |
| gamma-linolenic acid + eicosapentaenoic acid | | |
| alpha-linolenic acid + docosahexaenoic acid + | diindolylmethane | rosiglitazone |
| gamma-linolenic acid + eicosapentaenoic acid | | |
| alpha-linolenic acid + docosahexaenoic acid + | diindolylmethane | pioglitazone |
| gamma-linolenic acid + eicosapentaenoic acid | | |
| alpha-linolenic acid + docosahexaenoic acid + | diindolylmethane | muraglitizar |
| gamma-linolenic acid + eicosapentaenoic acid | | |
| alpha-linolenic acid + docosahexaenoic acid + | diindolylmethane | naveglitizar |
| gamma-linolenic acid + eicosapentaenoic acid | | |

In an alternative exemplary embodiment, folic acid may be combined with any of the combinations delineated in Table F.

In each of the combinations, the amount of polyunsaturated fatty acid in the combination may range from about 10 mg to about 10,000 mg, and the amount of diindolylmethane in the combination may range from about 10 mg to about 10,000 mg. In an alternative embodiment, the amount of polyunsaturated fatty acid in the combination may range from about 250 mg to about 2500 mg, and the amount of diindolylmethane in the combination may range from about 25 mg to about 1000 mg. In an additional alternative embodiment, the amount of polyunsaturated fatty acid in the combination may range from about 500 mg to about 1000 mg, and the amount of diindolylmethane in the combination may range from about 50 mg to about 500 mg. For each of the embodiments, when folio acid is present in the combination, the amount may range from about 0.1 mg to about 15 mg. In an alternative embodiment, the amount of folio acid in the combination may range from about 1 mg to about 10 mg. In yet an additional embodiment, the amount of folic acid in the combination may range from about 1 mg to about 5 mg.

### (III) Pharmaceutical Compositions

The combinations are generally formulated into a pharmaceutical composition that may be manufactured in one or several dosage forms. Suitable dosage forms include a tablet, including a suspension tablet, a chewable tablet, an effervescent tablet or caplet; a pill; a powder such as a sterile packaged powder, a dispensable powder, and an effervescent powder; a capsule including both soft or hard gelatin capsules such as HPMC capsules; a lozenge; a sachet; a sprinkle; a reconstitutable powder or shake; a troche; pellets; granules; liquids; suspensions; emulsions; or semisolids and gels. Alternatively, the pharmaceutical compositions may be incorporated into a food product or powder for mixing with a liquid, or administered orally after mixing with a non-foodstuff liquid.

The pharmaceutical compositions, in addition to being suitable for administration in multiple dosage forms, may also be administered with various dosage regimens. It is contemplated that the ingredients forming the various pharmaceutical compositions of the invention may be formulated into the same dosage form or in separate dosage forms and included in a variety of packaging options. The dosage forms may also be bi-daily, weekly, bi-weekly, monthly, or bimonthly dosages of any of the ingredients. Typically, the dosage form will provide a daily dosage. The different dosage forms may be packaged separately or they may in be included within the same package contained in different cavities, such as in a strip pack or a blister pack.

The amount and types of ingredients (i.e., polyunsaturated fatty acid, diindolylmethane, hormone, vitamin, etc), and other excipients useful in each of these dosage forms are described throughout the specification. It should be recognized that where a combination of ingredients and/or excipient, including specific amounts of these components, is described with one dosage form that the same combination could be used for any other suitable dosage form. Moreover, it should be understood that one of skill in the art would, with the teachings found within this application, be able to make any of the dosage forms listed above by combining the amounts and types of ingredients administered as a combination in a single dosage form, or as separate dosage forms administered together, as described in the different sections of the specification.

It is contemplated that, if appropriate, one or more of the ingredients forming the pharmaceutical composition of the present invention can exist in tautomeric, geometric or stereoisomeric forms without departing from the scope of the invention. The present invention contemplates all such compounds, including cis- and trans-geometric isomers, E- and Z-geometric isomers, R- and S-enantiomers, diastereomers, d-isomers, I-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures of R and S forms for each stereocenter present.

Moreover, one or more of the ingredients forming the pharmaceutical composition of the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine-(N-methylglucamine) and procaine. All of these salts may be prepared by conventional means from the corresponding compound by reacting, for example, the appropriate acid or base with the one or more of the corresponding compounds set forth herein.

The particle size of the ingredients forming the pharmaceutical composition may be an important factor that can effect bioavailability, blend uniformity, segregation, and flow properties. In general, smaller particle sizes of a drug increase its bioabsorption rate by increasing the surface area. The particle size of the drug and excipients can also affect the suspension properties of the pharmaceutical formulation. For example, smaller particles are less likely to settle and therefore form better suspensions. In various embodiments, the average particle size of the dry powder of the various ingredients (which can be administered directly, as a powder for suspension, or used in a solid dosage form) is less than about 500 microns in diameter, or less than about 450 microns in diameter, or less than about 400 microns in diameter, or less than about 350 microns in diameter, or less than about 300 microns in diameter, or less than about 250 microns in diameter, or less than about 200 microns in diameter, or less than about 150 microns in diameter, or less than about 100 microns in diameter, or less than about 75 microns in diameter, or less than about 50 microns in diameter, or less than about 25 microns in diameter, or less than about 15 microns in diameter. In some applications the use of particles less than 15 microns in diameter may be advantageous. In these cases colloidal or nanosized particles in the particle size range of 15 microns down to 10 nanometers may be advantageously employed.

The pharmaceutical compositions of the present invention can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, extruding, coacervation and the like.

(IV) Methods of Treatment and/or Prevention

It is believed, without being bound by any particular theory, that the combinations of the invention beneficially alter the metabolism of hormones, such as androgens and estrogen. In particular, the combinations alter the metabolism of estrogen in the subject such that there is an increase in the metabolism of estrogen through the 2-hydroxy pathway, and a decrease in estrogen metabolism through the 16-hydroxy pathway and the 4-hydroxy pathway. By altering estrogen metabolism in this manner, more “good estrogen” metabolites are produced (i.e., 2-hydroxy estrone and 2-methoxyestrone) and less “bad estrogen” metabolites are produced (i.e., 16-hydroxy estrone and 4-hydroxy estrone). Generally speaking, good estrogen metabolites function as antioxidants, and can facilitate the elimination of damaged or cancerous cells. In contrast, bad estrogen metabolites generate reactive oxygen species, and concomitantly, promote cancer.

An aspect of the invention, accordingly, is the use of the combinations to alter the pathway of estrogen metabolism to favor production of 2-hydroxy estrone metabolites. In one embodiment, production of 2-hydroxy estrogen metabolites is increased from about 1% to about 99%. In other embodiments, production of 2-hydroxy estrone metabolites is increased by at least 5%, by at least 10%, by at least 15%, by at least 20%, by at least 25%, by at least 30%, by at least 35%, by at least 40%, by at least 45%, by at least 50%, by at least 60%, by at least 65%, by at least 70%, by at least 75%, by at least 80%, by at least 85%, by at least 90%, by at least 95%, or by greater than about 99%. Increases in the production of 2-hydroxy estrone metabolites may be measured by any methods generally known in the art, such as by mass spectrometry or by gas chromatography.

The combinations may be used in female subjects to provide a more beneficial hormone replacement therapy. Traditional estrogen replacement therapy has benefits and detriments. Its benefits include relief from symptoms associated with menopause (e.g., hot flashes and vaginal dryness), and protection against osteoporosis. In contrast, its drawbacks include an increased risk of breast cancer, heart disease, and stroke. These drawbacks generally result from metabolism of estrogen through the 16-hydroxy pathway and the 4-hydroxy pathway. Advantageously, the combinations of the invention may be utilized to alleviate the aforementioned drawbacks of hormone replacement therapy while maintaining its benefits by altering the pathway of estrogen metabolism to favor production of 2-hydroxy estrone metabolites, as discussed above.

It is also contemplated that the combinations may be utilized to enhance apoptotic activity in aberrant or damaged cells. In this context, a combination of agents is generally suitable if it increases the number of normal cells and/or decreases the number of aberrant cells in a subject. Aberrant cells include, for example, precancerous cells, such as cells undergoing dysplasia or hyperplasia, and all stages of cancer cells. The number of aberrant cells may be decreased by about 5%, about 10%, about 15%, about 20%, about 25%, about 30%, about 35%, about 40%, about 45%, about 50%, about 55%, about 60%, about 65%, about 70%, about 75%, about 80%, about 85%, or by greater than about 90%. Determination of an increase or a decrease in normal cells or aberrant cells may be conducted in vitro or in vivo by methods generally known in the art.

The combinations are also useful for the treatment and/or prevention of several types of cancer in a subject. In one alternative, the cancer is an estrogen related cancer. Examples of estrogen related cancers include breast cancer, cervical cancer, uterine cancer, vaginal cancer, and ovarian cancer. Alternatively, the cancer may be an androgen related cancer. Examples of androgen related cancers include prostate cancer, and testicular cancer.

In yet another aspect, the combinations may be used to treat and/or prevent indications other than cancer. In one embodiment, the combination may be used to treat and/or prevent inflammation. In another embodiment, the combination may be used to treat and/or prevent cardiovascular disease. In still another embodiment, the combinations may be used to treat and/or prevent cerebrovascular disease. In another embodiment, the combinations may be used to treat a microbial infection, such as a viral infection, bacterial infection, or fungal infection. In yet another embodiment, the combinations may be used to treat arthritis, such as rheumatoid arthritis and osteoarthritis.

A wide range of subjects may be benefited by the combinations of the invention. In an exemplary embodiment, the subject is a human. In other embodiments, the subject is a companion animal such as a cat, dog, or horse. Alternatively, the subject may be agricultural animals, such as beef cattle, dairy cattle, swine, and sheep.

All publications, patents, patent applications and other references cited in this application are herein incorporated by reference in their entirety as if each individual publication, patent, patent application or other reference were specifically and individually indicated to be incorporated by reference.

1. A combination, the combination comprising a polyunsaturated fatty acid and diindolylmethane.

2. The combination of claim 1, wherein the polyunsaturated fatty acid is an omega fatty acid.

3. The combination of claim 1, wherein the polyunsaturated fatty acid is an omega-3 fatty acid.

4. The combination of claim 3, wherein the omega-3 fatty acid is selected from the group consisting of all-cis 7,10,13-hexadecatrienoic acid; all-cis-9,12,15-octadecatrienoic acid; all-cis-6,9,12,15-octadecatetraenoic acid; all-cis-8,11,14,17-eicosatetraenoic acid; all-cis-5,8,11,14,17-eicosapentaenoic acid; all-cis-7,10,13,16,19-docosapentaenoic acid; all-cis-4,7,10,13,16,19-docosahexaenoic acid; all-cis-4,7,10,13,16,19-docosahexaenoic acid; and all-cis-6,9,12,15,18,21-tetracosenoic acid.

5. The combination of claim 1, wherein the polyunsaturated fatty acid is an omega-6 fatty acid.

6. The combination of claim 5, wherein the omega-6 fatty acid is selected from the group consisting of all-cis-9,12-octadecadienoic acid; all-cis-6,9,12-octadecatrienoic acid; all-cis-11,14-eicosadienoic acid; all-cis-8,11,14-eicosatrienoic acid; all-cis-5,8,11,14-eicosatetraenoic acid; all-cis-13,16-docosadienoic acid; all-cis-7,10,13,16-docosatetraenoic acid; and all-cis-4,7,10,13,16-docosapentaenoic acid.

7. The combination of claim 1, wherein the polyunsaturated fatty acid is an omega-9 fatty acid.

8. The combination of claim 7, wherein the omega-9 fatty acid is selected from the group consisting of cis-9-octadecenoic acid; cis-11-eicosenoic acid; all-cis-5,8,11-eicosatrienoic acid; cis-13-docosenoic acid; and cis-15-tetracosenoic acid.

9. The combination of claim 1, further comprising a vitamin selected from the group consisting of vitamin C, vitamin A, vitamin E, vitamin B12, vitamin K, riboflavin, niacin, vitamin D, vitamin B6, folic acid, pyridoxine, thiamine, pantothenic acid, and biotin.

10. The combination of claim 1, further comprising a mineral selected from the group consisting of calcium, chromium, copper, iodine, iron, magnesium, manganese, molybdenum, phosphorus, potassium, selenium, and zinc.

11. The combination of claim 1, further comprising a non-steroidal anti-inflammatory agent selected from the group consisting of salicylates; arylalkanoic acids; 2-arylpropionic acids; N-arylanthranilic acids; pyrazolidine derivatives; oxicams; cyclooxygenase-2 inhibitors; and sulphonanilides.

12. The combination of claim 11, wherein the non-steroidal anti-inflammatory agent is selected from the group consisting of aspirin, amoxiprin, benorilate, choline magnesium salicylate, diflunisal, faislamine, methyl salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, and nimesulide.

13. The combination of claim 1, further comprising a steroidal anti-inflammatory agent selected from the group consisting of dexamethasone, hydrocortisone, cortisone acetate, prednisone, prednisolone, methylprednisolone, betamethasone, triamcinolone, beclometasone, fludrocortisone acetate, fludrocortisone acetate, deoxycorticosterone acetate, and aldosterone.

14. The combination of claim 1, further comprising an agent selected from the group consisting of thiazolidinedione, PPAR-gamma ligands, exenatide, rosiglitazone, pioglitazone, muraglitizar, and naveglitizar.

15. The combination of claim 1, further comprising an HMG-CoA reductase inhibitor selected from the group consisting of mevastatin, atorvastatin, cerivastatin, fluvastatin, pitavastatin, rosuvastatin, simvastatin, lovastatin, and pravastatin.

16. The combination of claim 1, further comprising an anti-proliferative agent that acts by a mechanism selected from the group consisting of disruption of cellular microtubule structure, downregulation of angiogenesis, inhibition of VEGF, disruption of DNA replication, alkylation, disruption of endocrine function, inhibition of tyrosine kinase, inhibition of topoisomerase, inhibition of testosterone signal, inhibition of estrogen production, inhibition of estrogen signal, and upregulation of gonadotropin-releasing hormone.

17. The combination of claim 16, wherein the anti-proliferative agent is selected from the group consisting of taxanes, paclitaxel, docetaxel, Vinca alkaloids, vincristine, vinblastine, vinorelbine, vindesine, bevacizumab, tyrosine kinase inhibitors, imatnib, imatnib mesylate, gefitinib, erlotinib, cisplatin, carboplatin, oxaliplatin, mechlorethamine, cyclophosphamide, chlorambucil, azathioprine, mercaptopurine, irinotecan, topotecan hydrochloride, amsacrine, etoposide, etoposide phosphate, teniposide, finasteride, anastrozole, formestane, exemestane, vorozole, letrozole, tamoxifen, and goserelin.

18. The combination of claim 1, further comprising an anti-immune agent selected from the group consisting of immunosuppressants, monoclonal antibodies, erbB2 receptor, epidermal growth factor receptor, circulating VEGF ligand, cytokines, mTOR kinase inhibitor, CD20 receptor, and RTK inhibitor.

19. The combination of claim 1, further comprising an apoptotic agent selected from the group consisting of proteasome inhibitors, and DR4/DR5 receptor agonist.

20. The combination of claim 1, further comprising a hormone selected from the group consisting of estrogen; conjugated estrogens; dienestrol; estrone; esterified estrogens; estradiol; estriol; estropipate; and ethinyl estradiol.

21. The combination of claim 1, further comprising a hormone selected from the group consisting of testosterone, dehydroepiandrosterone, androstenedione, androstenediol, androsterone, and dihydrotestosterone.

22. The combination of claim 1, wherein the combination is a pharmaceutical composition.

23. The pharmaceutical composition of claim 22, further comprising at least one pharmaceutically acceptable excipient.

24. The pharmaceutical composition of claim 22, wherein the composition is formulated as a single dosage form.

25. The pharmaceutical composition of claim 22, wherein the composition is formulated into several dosage forms.

26. The pharmaceutical composition of claim 25, wherein each dosage form is packaged together in a blister pack.

27. The pharmaceutical composition of claim 22, wherein the dosage form is selected from the group consisting of a suspension tablet, a chewable tablet, an effervescent tablet, a pill, a dispensable powder, an effervescent powder, a soft capsule, a hard gelatin, a lozenge, a sachet, a sprinkle, a reconstitutable shake, a troche, a pellet, granules, liquids, suspensions, emulsions, semisolids, gels, and food products.

28. The combination of claim 1, wherein the amount of polyunsaturated fatty acid in the combination is from about 10 mg to about 10,000 mg, and the amount of diindolylmethane in the combination is from about 10 mg to about 10,000 mg.

29. The combination of claim 1, wherein the amount of polyunsaturated fatty acid in the combination is from about 250 mg to about 2500 mg, and the amount of diindolylmethane in the combination is from about 25 mg to about 1000 mg.

30. The combination of claim 1, wherein the amount of polyunsaturated fatty acid in the combination is from about 500 mg to about 1000 mg, and the amount of diindolylmethane in the combination is from about 50 mg to about 500 mg.

31. The combination of claim 1, wherein combination is a pharmaceutical composition formulated as a single dosage form; the polyunsaturated fatty acid is EFA in an amount greater than about 500 mg; and the amount of diindolylemethane is greater than about 200 mg.

32. The combination of claim 31, further comprising ALA in an amount greater than about 25 mg.

33. A combination, the combination comprising a polyunsaturated fatty acid, diindolylmethane, and folic acid.

34. The combination of claim 33, wherein the polyunsaturated fatty acid is an omega fatty acid.

35. The combination of claim 33, wherein the polyunsaturated fatty acid is an omega-3 fatty acid.

36. The combination of claim 35, wherein the omega-3 fatty acid is selected from the group consisting of all-cis 7,10,13-hexadecatrienoic acid; all-cis-9,12,15-octadecatrienoic acid; all-cis-6,9,12,15-octadecatetraenoic acid; all-cis-8,11,14,17-eicosatetraenoic acid; all-cis-5,8,11,14,17-eicosapentaenoic acid; all-cis-7,10,13,16,19-docosapentaenoic acid; all-cis-4,7,10,13,16,19-docosahexaenoic acid; all-cis-4,7,10,13,16,19-docosahexaenoic acid; and all-cis-6,9,12,15,18,21-tetracosenoic acid.

37. The combination of claim 33, wherein the polyunsaturated fatty acid is an omega-6 fatty acid.

38. The combination of claim 37, wherein the omega-6 fatty acid is selected from the group consisting of all-cis-9,12-octadecadienoic acid; all-cis-6,9,12-octadecatrienoic acid; all-cis-11,14-eicosadienoic acid; all-cis-8,11,14-eicosatrienoic acid; all-cis-5,8,11,14-eicosatetraenoic acid; all-cis-13,16-docosadienoic acid; all-cis-7,10,13,16-docosatetraenoic acid; and all-cis-4,7,10,13,16-docosapentaenoic acid.

39. The combination of claim 33, wherein the polyunsaturated fatty acid is an omega-9 fatty acid.

40. The combination of claim 39, wherein the omega-9 fatty acid is selected from the group consisting of 9Z,11E-octadeca-9,1-dienoic acid; 10E,12Z-octadeca-9,1-dienoic acid; 8E,10E,12Z-octadecatrienoic acid; 8E,10E,12E-octadecatrienoic acid; 8E,10Z,12E-octadecatrienoic acid; 9E,11E,13Z-octadeca-9,11,13-trienoic acid; 9E,11E,13E-octadeca-9,11,13-trienoic acid; 9Z,11Z,13E-octadeca-9,11,13-trienoic acid; and 9E,11Z,13E-octadeca-9,11,13-trienoic acid.

41. The combination of claim 33, further comprising a vitamin selected from the group consisting of vitamin C, vitamin A, vitamin E, vitamin B12, vitamin K, riboflavin, niacin, vitamin D, vitamin B6, folic acid, pyridoxine, thiamine, pantothenic acid, and biotin.

42. The combination of claim 33, further comprising a mineral selected from the group consisting of calcium, chromium, copper, iodine, iron, magnesium, manganese, molybdenum, phosphorus, potassium, selenium, and zinc.

43. The combination of claim 33, further comprising a non-steroidal anti-inflammatory agent selected from the group consisting of salicylates; arylalkanoic acids; 2-arylpropionic acids; N-arylanthranilic acids; pyrazolidine derivatives; oxicams; cyclooxygenase-2 inhibitors; and sulphonanilides.

44. The combination of claim 43, wherein the non-steroidal anti-inflammatory agent is selected from the group consisting of aspirin, amoxiprin, benorilate, choline magnesium salicylate, diflunisal, faislamine, methyl salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, and nimesulide.

45. The combination of claim 33, further comprising a steroidal anti-inflammatory agent selected from the group consisting of dexamethasone, hydrocortisone, cortisone acetate, prednisone, prednisolone, methylprednisolone, betamethasone, triamcinolone, beclometasone, fludrocortisone acetate, fludrocortisone acetate, deoxycorticosterone acetate, and aldosterone.

46. The combination of claim 33, further comprising an agent selected from the group consisting of thiazolidinedione, PPARγ ligands, exenatide, avendia, actos, muraglitizar, and naveglitizar.

47. The combination of claim 33, further comprising a HMG-CoA reductase inhibitor selected from the group consisting of mevastatin, compactin, atorvastatin, lipitor, sortis, torvast, torvacard, totalip, tulip, xarator, atorpic, liprimar, fluvastatin, lescol, canef, pitavastatin, itavastatin, itabavastin, nisvastatin, rosuvastatin, crestor, simvastatin, zocor, denan, liponorm, sinvacor, sivastin, lipovas, lodales, zocord, zimstat, simvahexyl, lipex, lovastatin, mevacor, advicor, altocor, altoprev, statosan, pravastatin, pravachol, and selektine.

48. The combination of claim 33, further comprising an anti-proliferative agent that acts by a mechanism selected from the group consisting of disruption of cellular microtubule structure, downregulation of angiogenesis, a VEGF inhibitor, disruption of DNA replication, an alkylating agent, disruption of endocrine function, tyrosine kinase inhibitors, type I topoisomerase inhibitors, type II topoisomerase inhibitors, testosterone signal inhibitor, estrogen production inhibitors, estrogen signal inhibitor, and gonadotropin-releasing hormone agonists.

49. The combination of claim 48, wherein the anti-proliferative agent is selected from the group consisting of taxanes, taxol, docetaxel, Vinca alkaloids, vincristine, vinblastine, vinorelbine, vindesine, avastin, tyrosine kinase inhibitors, gleevec, gefitinib, erlotinib, cisplatin, carboplatin, oxaliplatin, mechloethamine, cyclophosphamide, chlorambucil, azathioprine, mercaptopurine, irinotecan, topotecan, amsacrine, etoposide, etoposide phosphate, teniposide, etoposide, teniposide, finasteride, anastrozole, formestane, exemestane, vorozole, letrozole, tamoxifen, and goserelin.

50. The combination of claim 33, further comprising an anti-immune agent selected from the group consisting of immunosuppressants, monoclonal antibodies, erbB2 receptor, epidermal growth factor receptor, circulating VEGF ligand, cytokines, mTOR kinase inhibitor, CD20 receptor, and RTK inhibitor.

51. The combination of claim 33, further comprising an apoptotic agent selected from the group consisting of proteasome inhibitors, and DR4/DR5 receptor agonist.

52. The combination of claim 33, further comprising a hormone selected from the group consisting of estrogen; conjugated estrogens; dienestrol; estrone; esterified estrogens; estradiol; estriol; estropipate; and ethinyl estradiol.

53. The combination of claim 33, further comprising a hormone selected from the group consisting of testosterone, dehydroepiandrosterone, androstenedione, androstenediol, androsterone, and dihydrotestosterone.

54. The combination of claim 33, wherein the combination is a pharmaceutical composition.

55. The pharmaceutical composition of claim 54, further comprising at least one pharmaceutically acceptable excipient.

56. The pharmaceutical composition of claim 55, wherein the composition is formulated as a single dosage form.

57. The pharmaceutical composition of claim 55, wherein the composition is formulated into several dosage forms

58. The pharmaceutical composition of claim 57, wherein each dosage form is packaged together in a blister pack.

59. The pharmaceutical composition of claim 54, wherein the dosage form is selected from the group consisting of a suspension tablet, a chewable tablet, an effervescent tablet, a pill, a dispensable powder, an effervescent powder, a soft capsule, a hard gelatin, a lozenge, a sachet, a sprinkle, a reconstitutable shake, a troche, a pellet, granules, liquids, suspensions, emulsions, semisolids, gels, and food products.

60. The combination of claim 33, wherein the amount of polyunsaturated fatty acid in the combination is from about 10 mg to about 10,000 mg; the amount of diindolylmethane in the combination is from about 10 mg to about 10,000 mg; and the amount of folic acid in the combination is from about 0.1 mg to about 15 mg.

61. The combination of claim 33, wherein the amount of polyunsaturated fatty acid in the combination is from about 250 mg to about 2500 mg; the amount of diindolylmethane in the combination is from about 25 mg to about 1000 mg; and the amount of folic acid in the combination is from about 1 mg to about 10 mg.

62. The combination of claim 33, wherein the amount of polyunsaturated fatty acid in the combination is from about 500 mg to about 1000 mg; the amount of diindolylmethane in the combination is from about 50 mg to about 500 mg; and the amount of folic acid in the combination is from about 1 mg to about 5 mg.

63. The combination of claim 33, wherein combination is a pharmaceutical composition formulated as a single dosage form; the polyunsaturated fatty acid is EFA in an amount greater than about 500 mg; the amount of diindolylemethane is greater than about 200 mg; and the amount of folic acid is greater than about 1 mg.

64. The combination of claim 63, further comprising ALA in an amount greater than about 25 mg.

65. A method for increasing the number of normal cells and decreasing the number of aberrant cells in a subject, the method comprising administering to the subject a combination comprising a polyunsaturated fatty acid and diindolylmethane.

66. The method of claim 65, further comprising administering folic acid.

67. The method of claim 65 wherein the polyunsaturated fatty acid is an omega fatty acid.

68. The method of claim 67, wherein the omega fatty acid is selected from the group consisting of an omega-3 fatty acid, an omega-6 fatty acid, and an omega-9 fatty acid.

69. The method of claim 65 further comprising at least one agent selected from the group consisting of a vitamin, a mineral, a non-steroidal anti-inflammatory agent, a steroidal anti-inflammatory agent, thiazolidinedione, an HMG-CoA reductase, an anti-proliferative agent, an anti-immune agent, an apoptotic agent, and a hormone.

70. The method of claim 65 wherein the combination is a pharmaceutical composition.

71. The method of claim 70, further comprising at least one pharmaceutically acceptable excipient.

72. The method of claim 71, wherein the pharmaceutical composition is formulated as a single dosage form.

73. The method of claim 71, wherein the pharmaceutical composition is formulated into several dosage forms.

74. The method of claim 73, wherein each dosage form is packaged together in a blister pack.

75. The method of claim 71, wherein the dosage form is selected from the group consisting of a tablet, a pill, a powder, a capsule, a lozenge, a sachet, a sprinkle, a troche, and a pellet, granules, liquids, suspensions, emulsions, semisolids, gels, and food products.

76. The method of claim 65, wherein the amount of polyunsaturated fatty acid in the combination is from about 10 mg to about 10,000 mg, and the amount of diindolylmethane in the combination is from about 10 mg to about 10,000 mg.

77. The method of claim 65, wherein the amount of polyunsaturated fatty acid in the combination is from about 250 mg to about 2500 mg, and the amount of diindolylmethane in the combination is from about 25 mg to about 1000 mg.

78. The method of claim 65, wherein the amount of polyunsaturated fatty acid in the combination is from about 500 mg to about 1000 mg, and the amount of diindolylmethane in the combination is from about 50 mg to about 500 mg.

79. The method of claim 65, wherein combination is a pharmaceutical composition formulated as a single dosage form; the polyunsaturated fatty acid is EFA in an amount greater than about 50 mg; and the amount of diindolylemethane is greater than about 25 mg.

80. The method of claim 79, further comprising ALA in an amount greater than about 25 mg.

81. The method of claim 76 further comprising folic acid in an amount from about 0.1 mg to about 15 mg.

82. The method of claim 76 further comprising folic acid in an amount not less than 1 mg.

83. The method of claim 65, wherein an aberrant cell is selected from the group consisting of a precancerous cell and a cancerous cell.

84. The method of claim 65, wherein the increase in the number of normal cells and the decrease in the number of aberrant cells reduces the risk that the subject will develop an indication selected from the group consisting of cancer, inflammation, cardiovascular disease, cerebrovascular disease, viral infections, rheumatoid arthritis, and osteoarthritis.

85. The method of claim 84, wherein the cancer is an estrogen related cancer selected from the group consisting of breast cancer, cervical cancer, uterine and ovarian cancer.

86. The method of claim 84, wherein the cancer is an androgen related cancer selected from the group consisting of prostate cancer, and testicular cancer.

87. The method of claim 65, wherein the combination alters the metabolism of estrogen in the subject such that there is an increase in the metabolism of estrogen through the 2-hydroxy pathway, and a decrease in estrogen metabolism through the 16-hydroxy pathway and the 4-hydroxy pathway.

88. A combination comprising from about 250 mg to about 1000 mg of diindolylmethane and from about 1 mg to about 5 mg of folic acid.

89. The combination of claim 88, further comprising at least one agent selected from the group consisting of a vitamin, a mineral, a non-steroidal anti-inflammatory agent, a steroidal anti-inflammatory agent, thiazolidinedione, an HMG-CoA reductase, an anti-proliferative agent, an anti-immune agent, an apoptotic agent, and a hormone.

90. A combination consisting of diindolylmethane and folic acid.

91. The combination of claim 32 or claim 64, wherein the amount of ALA in the combination is from about 50 mg to about 5000 mg.

92. The combination of claim 32 or claim 64, wherein the amount of ALA in the combination is from about 100 mg to about 3000 mg.

93. The combination of claim 32 or claim 64, wherein the amount of ALA in the combination is from about 200 mg to about 1000 mg.

94. The combination of claim 32 or claim 64, wherein the amount of ALA in the combination is from about 300 mg to about 600 mg.

95. The method of claim 79, wherein the amount of EFA in the combination is from about 50 mg to about 3,000 mg, and the amount of diindolylmethane in the combination is from about 25 mg to about 750 mg.

96. The method of claim 79, wherein the amount of EFA in the combination is from about 100 mg to about 2,000 mg, and the amount of diindolylmethane in the combination is from about 100 mg to about 500 mg.

97. The method of claim 79, wherein the amount of EFA in the combination is from about 200 mg to about 1,000 mg, and the amount of diindolylmethane in the combination is from about 150 mg to about 450 mg.

98. The method of claim 79, wherein the amount of EFA in the combination is from about 300 mg to about 750 mg, and the amount of diindolylmethane in the combination is from about 200 mg to about 300 mg.

99. The method of claim 80, wherein the amount of ALA in the combination is from about 50 mg to about 5000 mg.

100. The method of claim 80, wherein the amount of ALA in the combination is from about 100 mg to about 3000 mg.

101. The method of claim 80, wherein the amount of ALA in the combination is from about 200 mg to about 1000 mg.

102. The method of claim 80, wherein the amount of ALA in the combination is from about 300 mg to about 600 mg.
